(19) United States
(12) Patent Application Publication
(10) Pub. No.: US 2018/0280437 A1
(43) Pub. Date: Oct. 4, 2018

(54) CHIMERIC ANTIGEN RECEPTORS FOR MELANOMA AND USES THEREOF

(71) Applicant: KITE PHARMA, INC., Santa Monica, CA (US)

(72) Inventors: Jed J.W. Wiltzius, Santa Monica, CA (US); Stuart A. Sievers, Santa Monica, CA (US)

(21) Appl. No.: 15/919,408

(22) Filed: Mar. 13, 2018

Related U.S. Application Data
(60) Provisional application No. 62/710,561, filed on Feb. 16, 2018.
(60) Provisional application No. 62/470,703, filed on Mar. 13, 2017.

(51) Int. Cl. A61K 35/17; A61K 39/00; A61P 35/00

(52) U.S. Cl. CPC A61K 35/17; A61K 39/0011; A61K 2039/5158; A61K 2039/505; A61K 2039/5156; A61P 35/00

(57) ABSTRACT

The present invention relates to Chimeric Antigen Receptors (CARs) comprising antigen binding domains that specifically bind melanoma cells, polynucleotides encoding such CARs, and vectors comprising such polynucleotides. The present invention further relates to engineered cells comprising such polynucleotides and/or transduced with such viral vectors, and compositions including a plurality of engineered T cells. The present invention also relates to methods for manufacturing such engineered T cells and compositions and uses in treating a melanoma such engineered T cells and compositions.

### CROSS-REFERENCE TO RELATED APPLICATIONS

This application claims priority to U.S. Provisional Patent Application Ser. No. 62/470,703, filed Mar. 13, 2017 and to U.S. Provisional Patent Application Ser. No. 62/710,561, filed Feb. 16, 2018, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 30, 2018, is named KPI-001US1_SL.txt and is 111,356 bytes in size.

### BACKGROUND

Melanoma is a type of skin cancer that develops from pigment-containing cells known as melanocytes. The American Cancer Society estimates that in the United States in 2017, 87,110 new cases of melanoma will be diagnosed and about 9,730 people will die from melanoma. If melanoma is not recognized and treated early, the cancer can advance and spread away from the skin surface and throughout a patient's body, where it becomes harder to treat and may be fatal.

Current therapies for melanoma include surgery, chemotherapy, radiation therapy, and cancer immunotherapy, which use a patient's own immune system to help fight the cancer. An example of latter treatment is the use of genetically-engineered T cell receptors (TCRs) which recognize cancer-related proteins and activate mechanisms that kill cancer cells. However, such TCRs rely on the cancer cell processing and displaying tumor antigens, presented by specific major histocompatibility complex (MHC) molecules, on the cancer cell's surface. If the quantity of processed and displayed tumor antigens is insufficient to activate the TCR-related mechanisms that kill cancer cells or if a patient lacks the necessary MHC allele, then the cancer cell may avoid being killed.

Accordingly, a need exists for novel and improved therapies for treating melanoma which do not rely on a cancer cell's processing and displaying of tumor antigens via MHC complexes.

### SUMMARY

The present invention addresses this need, and other needs, by providing compositions and methods comprising genetically engineered immune cells that specifically target and kill melanoma cells independent of MHC presentation. The invention is based in part upon the observation that the MART-1 antigen, which was known to be highly expressed in melanoma cells, possesses an extracellular domain; this extracellular domain is presented independent of the melanoma cell's processing and display of antigens on its surface. Independence from the cell's antigen display is a significant advantage of the present invention, since MART-1 is currently only shown to be displayed by one MHC allele (i.e., HLA-A2) which is only common in a fraction of Caucasian populations, whereas about 95% of melanoma patients have extracellular presentation of MART-1. See, e.g., Busam et al., Am. J. Surg. Pathol. 22(8): 976-82 (1998).

As described in more detail below, including the Examples section, certain anti-MART-1 antibodies have been determined to recognize and bind an extracellular epitope of MART-1 and derivatives of these antibodies may be used in antigen binding domains in chimeric antigen receptors (CARs). Polynucleotides encoding such CARs can be transduced and the CARs expressed in T cells, e.g., a patient's own T cells. When the transduced T cells are transplanted back to a patient, the CARS direct the T cells to recognize and bind an extracellular epitope of MART-1, which is highly presented on the surface of melanoma cells; thus, allowing binding of melanoma cells rather than non-cancerous melanin-containing cells, where the surface presentation of the MART-1 epitopes appear to be less abundant. This binding leads to activation of cytolytic mechanisms in the T cell that specifically kill the bound melanoma cells. Prior to the present invention, MART-1's extracellular domain has not been considered as a useful target for a CAR, which is capable of specifically killing melanoma cells. Thus, the present invention satisfies an unmet need that exists for novel and improved therapies for treating melanoma.

An aspect of the present invention is polynucleotide encoding a chimeric antigen receptor (CAR), wherein the CAR comprises at least an antigen binding domain, an activation domain, and a co-stimulatory domain, wherein the antigen binding domain is specific to MART-1.

In some embodiments, the antigen binding domain is specific to an extracellular epitope of MART-1.

In some embodiments, the antigen binding domain comprises an antibody or an antigen binding fragment thereof. The antibody or the antigen binding fragment thereof may be selected from the group consisting of an IgG, an Fab, an Fab′, an F(ab′)2, an Fv, an scFv, and a single-domain antibody (dAB). In some embodiments, the antibody or antigen binding fragment thereof is an scFv.

In some embodiments, the scFv comprises at least a light chain variable (VL) region and at least a heavy chain variable (VH) region. In some embodiments, the VH region is N-terminal to the VL region. In other embodiments, the VL region is N-terminal to the VH region.

In some embodiments, the VL region comprises a VL complementarity determining region (CDR) 1 (VL CDR1), a VL CDR2, and a VL CDR3 and the VH region comprises a VH CDR1, a VL CDR2, and a VL CDR3.

In some embodiments, the VL CDR1 is at least 90% identical to SEQ ID NO: 1, the VL CDR2 is at least 90% identical to SEQ ID NO: 2, and the VL CDR3 is at least 90% identical to SEQ ID NO: 3.

In some embodiments, the VH CDR1 is at least 90% identical to SEQ ID NO: 7 or 10, the VH CDR2 is at least 90% identical to SEQ ID NO: 8 or 11, and the VH CDR3 is at least 90% identical to SEQ ID NO: 9.

In some embodiments, the VL is at least 85% identical to SEQ ID NO: 18.

In some embodiments, the VH is at least 85% identical to SEQ ID NO: 19.

In some embodiments, the antigen binding domain is at least 80% identical to SEQ ID NO: 20.

In some embodiments, the antigen binding domain is at least 80% identical to SEQ ID NO: 21.

In some embodiments, the VL is encoded by a polynucleotide that is at least 85% identical to SEQ ID NO: 26.

In some embodiments, the VH is encoded by a polynucleotide that is at least 85% identical to SEQ ID NO: 27.

In some embodiments, the antigen binding domain is encoded by a polynucleotide that is at least 80% identical to SEQ ID NO: 28.

In some embodiments, the antigen binding domain is encoded by a polynucleotide that is at least 80% identical to SEQ ID NO: 29.

In some embodiments, the VL CDR1 is at least 90% identical to SEQ ID NO: 4, the VL CDR2 is at least 90% identical to SEQ ID NO: 5, and the VL CDR3 is at least 90% identical to SEQ ID NO: 6.

In some embodiments, the VH CDR1 is at least 90% identical to SEQ ID NO: 12, 15, or 17, the VH CDR2 is at least 90% identical to SEQ ID NO: 13 or 16, and the VH CDR3 is at least 90% identical to SEQ ID NO: 14.

In some embodiments, the VL is at least 85% identical to SEQ ID NO: 22.

In some embodiments, the VH is at least 85% identical to SEQ ID NO: 23.

In some embodiments, the antigen binding domain is at least 80% identical to SEQ ID NO: 24.

In some embodiments, the antigen binding domain is at least 80% identical to SEQ ID NO: 25.

In some embodiments, the VL is encoded by a polynucleotide that is at least 85% identical to SEQ ID NO: 30.

In some embodiments, the VH is encoded by a polynucleotide that is at least 85% identical to SEQ ID NO: 31.

In some embodiments, the antigen binding domain is encoded by a polynucleotide that is at least 80% identical to SEQ ID NO: 32.

In some embodiments, the antigen binding domain is encoded by a polynucleotide that is at least 80% identical to SEQ ID NO: 33.

In some embodiments, the CAR comprises a linker between domains. In some embodiments, the linker is GGGGS (SEQ ID NO: 72), GSG or AAA. In some embodiments, the linker comprises sequential repeats of GGGGS (SEQ ID NO: 72), GSG or AAA. In some embodiments, the linker comprises two or more sequential repeats of GGGGS (SEQ ID NO: 72), GSG or AAA. In some embodiments, the linker comprises three, four, or five sequential repeats of GGGGS (SEQ ID NO: 72), GSG or AAA. A table of representative linker sequences that can be used in various embodiments follows:

Table of Representative
Linker Sequences

GSTSGSGKPGSGEGSTKG (SEQ ID NO: 73)
GGGGSGGGGSGGGGS (SEQ ID NO: 74)
xxxGKPGSGExxxGKPGSGExxx
wherein x is any amino acid (SEQ ID NO: 75)
KPGSGE (SEQ ID NO: 76)
GKPGSGE (SEQ ID NO: 77)
GKPGSGG (SEQ ID NO: 78)
GGGSGKPGSGEGGGS (SEQ ID NO: 79)
GGGSGKPGSGEGGGGS (SEQ ID NO: 80)
GGGGSGKPGSGGGGS (SEQ ID NO: 81)
GGGGSGKPGSGEGGS (SEQ ID NO: 82)
GGGGSGKPGSGEGGGS (SEQ ID NO: 83)
GGGGSGKPGSGEGGGGS (SEQ ID NO: 84)
GSGKPGSGEG (SEQ ID NO: 85)
GKPGSGEG (SEQ ID NO: 86)
SGKPGSGE (SEQ ID NO: 87)
KPGSG (SEQ ID NO: 88)
STSGSGKPGSGEGST (SEQ ID NO: 89)
GGGGSGGGGSGGGGSG (SEQ ID NO: 90)
GGGGGSGGGGSGGGGS (SEQ ID NO: 91)
GGGGSGGGGSGGGGGS (SEQ ID NO: 92)

In some embodiments, there is a hinge domain between the activation domain and the co-stimulatory domain. In some embodiments, the CAR comprises a linker between the antigen binding domain and the hinge domain.

In some embodiments, the costimulatory domain and the hinge domain comprise a single contiguous domain.

Another aspect of the present invention is a vector comprising a polynucleotide of an above embodiment.

In some embodiments, the vector is an adenoviral vector, an adenovirus-associated vector, a DNA vector, a lentiviral vector, a plasmid, a retroviral vector, or an RNA vector.

In some embodiments, the vector is a retroviral vector, e.g., a lentiviral vector.

Yet another aspect of the present invention is a chimeric antigen receptor (CAR) encoded by a polynucleotide of an above embodiment or a vector of an above embodiment.

In another aspect, the present invention is a cell comprising a polynucleotide of an above embodiment, a vector of an above embodiment, or a chimeric antigen receptor (CAR) of an above an above embodiment.

In some embodiments, the cell is a T cell, e.g., an allogeneic T cell, an autologous T cell, an engineered autologous T cell (eACT™), or a tumor-infiltrating lymphocyte (TIL). In some embodiments, the T cell is a CD4+ T cell or a CD8+ T cell.

In some embodiments, the cell is an in vitro cell.

In some embodiments, the T cell is an autologous T cell.

In some embodiments, the cell produces at least Interferon gamma (IFNγ) upon activation by MART-1.

An aspect of the present invention is a composition comprising a plurality of cells of an above embodiment.

In some embodiments, the composition comprises CD4+ or CD8+ cells, e.g., CD4+ and CD8+ cells.

In some embodiments, each cell in the plurality of cells is an autologous T cell.

In some embodiments, the composition comprises at least one pharmaceutically-acceptable excipient.

Another aspect of the present invention is a composition comprising a polynucleotide of an above embodiment, a vector of an above embodiment, or a chimeric antigen receptor (CAR) of an above embodiment.

Yet another aspect of the present invention is a method for manufacturing a cell expressing a chimeric antigen receptor (CAR), comprising a step of transducing a cell with a polynucleotide of an above embodiment or a vector of an above embodiment.

In some embodiments, the cell is a lymphocyte, e.g., a natural killer cell, a T cell, or a B cell, isolated from a patient in need of treatment.

In some embodiments, the method further comprises a step of culturing the cell under conditions that promote cellular proliferation and/or T cell activation.

In some embodiments, the method further comprise a step of isolating desired T cells, e.g., after about six days of culturing.

In some embodiments, the desired T cells express CD4+ and/or CD8+.

In another aspect, the present invention is a method for treating melanoma comprising administering to a subject in need thereof a cell of an above embodiment or a composition of an above embodiment.

In yet another aspect, the present invention is a method for treating melanoma comprising administering to a subject in need thereof a cell expressing a chimeric antigen receptor (CAR) that specifically targets MART-1.

In some embodiments, the CAR comprises at least an antigen binding domain, an activation domain, and a co-stimulatory domain, wherein the antigen binding domain specifically binds to MART-1.

In some embodiments, the antigen binding domain specifically binds to an extracellular epitope of MART-1.

In some embodiments, the antigen binding domain is, is obtained from, or is derived from an IgG, an Fab, an Fab′, an F(ab′)2, an Fv, an scFv, or a single-domain antibody (dAB).

In some embodiments, the antigen binding domain is, is obtained from, or is derived from an scFv.

In some embodiments, the scFv comprises at least a light chain variable (VL) region and at least a heavy chain variable (VH) region.

In some embodiments, the VH region is N-terminal to the VL region or the VL region is N-terminal to the VH region.

In some embodiments, the CAR comprises a linker between domains. In some embodiments, the linker is GGGGS (SEQ ID NO: 72), GSG or AAA. In some embodiments, the linker comprises sequential repeats of GGGGS (SEQ ID NO: 72), GSG or AAA. In some embodiments, the linker comprises two or more sequential repeats of GGGGS (SEQ ID NO: 72), GSG or AAA. In some embodiments, the linker comprises three, four, or five sequential repeats of GGGGS (SEQ ID NO: 72), GSG or AAA and other embodiments disclosed in Table 1. In some embodiments, there may be a hinge domain between the activation domain and the co-stimulatory domain. In some embodiments, the CAR comprises a linker between the antigen binding domain and the hinge domain.

In some embodiments, the costimulatory domain and the hinge domain comprise a single contiguous domain.

In some embodiments, the cell is a T cell, e.g., an allogeneic T cell, an autologous T cell, an engineered autologous T cell (eACT), or a tumor-infiltrating lymphocyte (TIL).

In some embodiments, the T cell is a CD4+ T cell.

In some embodiments, the T cell is a CD8+ T cell.

In some embodiments, the cell is an in vitro cell.

In some embodiments, the T cell is an autologous T cell.

In some embodiments, the T cell produces at least Interferon gamma (IFNγ) upon activation by MART-1.

Generally, the present invention relates to Engineered Autologous Cell Therapy, abbreviated as “eACT™,” also known as adoptive cell transfer. eACT™, is a process by which a patient's own T cells are collected and subsequently genetically engineered to recognize and target one or more antigens expressed on the cell surface of one or more specific tumor cells or malignancies. See, FIG. 1A, FIG. 1B, and FIG. 2. T cells may be engineered to express, for example, a chimeric antigen receptor (CAR). CAR positive (CAR+) T cells are engineered to express a CAR. CARs may comprise, e.g., an extracellular single chain variable fragment (scFv) with specificity for a particular tumor antigen, which is directly or indirectly linked to an intracellular signaling part comprising at least one costimulatory domain, which is directly or indirectly linked to at least one activating domain; the components may be arranged in any order. The costimulatory domain may be derived from, e.g., CD28, and the activating domain may be derived from, e.g., any form of CD3-zeta. In some embodiments, the CAR is designed to have two, three, four, or more costimulatory domains. In some embodiments, a CAR is engineered such that the costimulatory domain is expressed as a separate polypeptide chain. Examples of CAR T cell therapies and constructs are described in U.S. Patent Publication Nos. 2013/0287748, 2014/0227237, 2014/0099309, and 2014/0050708; International Patent Publications Nos. WO2012033885, WO2012079000, WO2014127261, WO2014186469, WO2015080981, WO2015142675, WO2016044745, and WO2016090369; and Sadelain et al, Cancer Discovery, 3: 388-398 (2013), each of which are incorporated by reference in its entirety.

Any aspect or embodiment described herein may be combined with any other aspect or embodiment as disclosed herein. While the present invention has been described in conjunction with the detailed description thereof, the foregoing description is intended to illustrate and not limit the scope of the present invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts and scientific literature cited herein are hereby incorporated by reference.

Other features and advantages of the invention will be apparent from the Drawings and the following Detailed Description, including the Examples, and the claims.

### BRIEF DESCRIPTION OF THE FIGURES

The above and further features will be more clearly appreciated from the following detailed description when taken in conjunction with the accompanying drawings. The drawings however are for illustration purposes only; not for limitation.

FIG. 1A and FIG. 1B are cartoons depicting features of chimeric antigen receptor (CAR) manufacture and use. FIG. 1A shows an exemplary polynucleotide encoding a CAR, a viral vector comprising the CAR-encoding polynucleotide, transduction of the viral vector into a patient's T cell, integration into the host genome, and expression of a CAR on the surface of the transduced (“CAR-engineered”) T cell. FIG. 1B shows a CAR-engineered T cell which has recognized a target antigen located on the surface of a cancer cell. Recognition and binding of the target antigen activates mechanisms in the T cell including cytolytic activity, cytokine release, and T cell proliferation; these mechanisms promote killing of the cancer cells.

FIG. 2 is a cartoon showing major steps performed during Engineered Autologous Cell Therapy (eACT™).

FIG. 3 includes a series of plots showing detection of CARs of the present invention expressed on the surface of T-cells, which were obtained from two donor subjects. The “Mock” plots were transduced with a vector that lacked a polynucleotide encoding a CAR. The “M7” and “M8” T cells were transduced with vectors comprising, respectively, the M7 and M8 polynucleotide, as described herein.

FIG. 4A and FIG. 4B include a series of bar graphs demonstrating cytolytic activity of CARs of the present invention. In FIG. 4A, the cell type targeted in the assay (293T) lacked the tumor antigen recognized by the CAR, exemplifying the specificity of these CAR designs. In FIG. 4B, the cell type targeted in the assay (SKMEL28) has surface expression of MART-1 that is recognized by the CAR. Cell death of the target cell is identified by a reduction in luciferase activity. The Mock, M7, and M8 plots are as described above in FIG. 3. The “M9” T cells were transduced with a vector comprising the M9 polynucleotide, as described herein.

FIG. 5A to FIG. 5D include a series of bar graphs showing the cytolytic activity of CAR-expressing T cells (of the present invention) which have are contacted with increasing numbers of target cells. In FIG. 5A and FIG. 5B the cell type targeted in the assay (293T) lacked the tumor antigen recognized by the CAR, exemplifying the specificity of these CAR designs. In FIG. 5C and FIG. 5D, the cell type targeted in the assay (SKMEL28) has surface expression of MART-1 that is recognized by the CAR. Cell death of the target cell is identified by a reduction in luciferase activity. The “M7” and “M8” T cells were transduced with vectors comprising, respectively, the M7 and M8 polynucleotide, as described herein. The “Mock” T-cells were transduced with a vector that lacked a polynucleotide encoding a CAR. The experiments were repeated for two T-cell donors, 5244 and 5273.

FIG. 6A and FIG. 6B include a series of bar graphs demonstrating cytolytic activity of CARs of the present invention. In FIG. 6A, the cell type targeted in the assay (293T) lacked the tumor antigen recognized by the CAR, exemplifying the specificity of these CAR designs. In FIG. 6B, the cell type targeted in the assay (SKMEL28) has surface expression of MART-1 that is recognized by the CAR. Cell death of the target cell is identified by a reduction in luciferase activity. The Mock, M1, and M2 plots show data for T cells transduced with CAR constructs incubated with 293T and SKMEL28 cells at a 4:1 effector to target ratio. Luminescence of viable cells is measured.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone) Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systéme International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

An “antigen” refers to any molecule that provokes an immune response or is capable of being bound by an antibody, an antibody fragment thereof, or an antigen binding domain. Those of skill in the art will readily understand that any macromolecule, including virtually all proteins or peptides, can serve as an antigen. Generally, an antigen can be endogenously expressed, i.e. expressed by genomic DNA, or it can be recombinantly expressed, or it can be chemically synthesized. An antigen can be specific to a certain tissue, such as a cancer cell (e.g., a melanoma), or it can be broadly expressed. In addition, fragments of larger molecules can act as antigens. In some embodiments, antigens are tumor antigens. In some embodiments, the antigen is MART-1.

As used herein, the term “MART-1” (which is an acronym for “Melanoma Antigen Recognized by T cells 1”) is a protein that in humans is encoded by the MLANA or MELAN-A gene (an abbreviation for “melanocyte antigen”). MART-1 is also referred to as MLANA, MART1, melan-A, MLANA, antigen LB39-AA, antigen SK29-AA, and protein Melan-A. MART-1 is a putative 18 kDa protein consisting of 118 amino acids and having a single transmembrane domain. MART-1 expression is specific to pigment-producing cells, found in melanocytes within normal skin and the retina, but not in other normal tissues. It is a useful marker for melanocytic tumors (e.g., melanomas). MART-1 comprises a transmembrane domain which includes its amino acid residues number 27 to number 47 and a cytosolic domain which includes its amino acid residues number 48 to number 118. The trafficking of MART-1 through the plasma membrane during melanosome biosynthesis has been suggested previously (See, e.g., Chen et al., JBC, 287(29): 24082-91 (2012)); however, the epitope(s) that are presented at the cell surface have not been characterized in the literature.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen binding domain thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies may include, for example, both naturally occurring and non-naturally occurring (recombinantly-produced) antibodies, human, humanized, and non-human antibodies, monospecific antibodies, multi-specific antibodies (including bispecific antibodies), immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies (see, e.g., Stocks, (2004) Drug Discovery Today 9(22):960-66), heteroconjugate antibodies, single domain antibodies, monovalent antibodies, single chain antibodies or single-chain FVs (scFv), camelized antibodies, affybodies, Fab fragments, F(ab′)2 fragments, disulfide-linked FVs (sdFV), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments thereof. The term “antibody” includes both monoclonal antibodies and polyclonal antibodies.

Any antibody, fragment thereof, or amino acid sequence derived from an antibody and capable of recognizing and binding to MART-1, e.g., an extracellular epitope of MART-1, may be used in the present invention. The antibody may be non-commercially available or a commercially-available. Examples of commercially-available anti-MART-1 antibodies include, but are not limited to, the “A103” mouse monoclonal antibody (as described in U.S. Pat. No. 5,674,749); The “M2-72C10” mouse monoclonal antibody (e.g., Covance catalog number: SIG-38160); the “M2-9E3” mouse monoclonal antibody (e.g., Covance catalog number: SIG-38165); and the “EP1422Y” rabbit monoclonal antibody (e.g., Epitomics catalog number: 1989-1). Other commercially-available MART-1 antibodies may be used in the present invention. See, e.g., the World Wide Web (www) at antibodies-online.com.

An “amino acid sequence derived from an antibody” may be physically derived, e.g., expressed from a fragment of a polynucleotide encoding the antibody, or may be in silico derived, e.g., the nucleotide sequence determined to encode the antibody (or fragment thereof) is used to synthesize an artificial polynucleotide sequence (or fragment) and the artificial polynucleotide sequence is expressed as the antibody, or fragment thereof.

The term “extracellular domain of MART-1” refers to a portion of the MART-1 polypeptide that is presented outside of a cell such that the portion is capable of being recognized and bound by a chimeric antigen receptor (CAR) of the present invention. In some embodiments, the “extracellular domain of MART-1” may be an N-terminal portion of the MART-1 polypeptide. In some embodiments, the “extracellular domain of MART-1” may be a C-terminal portion of the MART-1 polypeptide. Similarly, a cell which “expresses MART-1 on its extracellular surface” comprises a portion of the MART-1 polypeptide that is presented on the cell's outside surface such that the portion is capable of being recognized and bound by a chimeric antigen receptor (CAR) of the present invention regardless of the means of trafficking that results in presentation of specific epitopes targeted by the CARs described herein.

“M1” and “M2”, as used herein, comprise antigen binding domain sequences or fragments thereof obtained from or modified from rabbit monoclonal antibodies synthesized by the “EP1422Y” hybridoma.

“M7”, “M8”, and “M9”, as used herein, comprise antigen binding domain sequences or fragments thereof obtained from or modified from mouse monoclonal antibodies synthesized by the “A103” hybridoma. The “M7” and “M8” CARs have scFvs as their antigen binding domains whereas the “M9” CAR has an Fab as its antigen binging domain.

An “antigen binding domain,” “antigen binding molecule,” “antigen binding portion,” “antibody,” “antibody fragment”, “antigen binding fragment (of an antibody)” refers to any molecule that comprises the antigen binding parts (e.g., CDRs) of the antibody from which the molecule (or amino acid sequence) is derived. An antigen binding domain may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding domains. Peptibodies (i.e., Fc fusion molecules comprising peptide binding domains) are another example of suitable antigen binding domains. In some embodiments, the antigen binding domain binds to an antigen on a tumor cell. In some embodiments, the antigen binding domain binds to an antigen on a cell involved in a hyperproliferative disease or to a viral or bacterial antigen. In some embodiments, the antigen binding domain binds to MART-1, e.g., an extracellular epitope of MART-1. In some embodiments, the antigen binding domain is an antibody fragment thereof, including one or more of the complementarity determining regions (CDRs) thereof.

In some embodiments, an antigen binding domain may be a natural binding partner for the antigen or a fragment of the natural binding partner. This would include Pmel17, a melanosomal protein that requires MART-1 binding for proper trafficking and function. For example, in nature, CD27 binds CD70 (also known as CD27L); thus, a CAR targeting CD70 may include CD27 or a fragment thereof as an antigen binding domain for binding CD70.

In some embodiments, the antigen binding domain comprises a single-chain variable fragment (scFv). An scFv is a fusion protein of the variable regions of the heavy (VH) and light chains (VL) of immunoglobulins, connected by a linker peptide (e.g., of about ten to about 25 amino acids). The linker is usually rich in glycine for flexibility, as well as serine or threonine for solubility. The linker may either connect the N-terminus of the VH with the C-terminus of the VL or connect the C-terminus of the VH with the N-terminus of the VL. This protein retains the specificity of the original immunoglobulin, despite removal of the constant regions and the introduction of the linker. An scFv may also include an N-terminal peptide sequence, which sometimes is referred to as a “signal peptide” or “leader sequence”.

An antigen binding domain is a component of a CAR which recognizes a target of interest (e.g., a cell expressing MART-1 on its plasma membrane). As used herein, in the context of a CAR of the present invention, an antigen binding domain means any component of a CAR that directs the CAR to a desired target and associates with that target. An antigen binding domain component of a CAR may comprise an scFv, which includes at least a heavy and light chain variable region joined by a linker. The heavy and light variable regions may be derived from the same antibody or two different antibodies. In some embodiments, an antigen binding domain used in a CAR includes the pairs of sequences comprising the amino acid sequences of SEQ ID NO: 18 and 19, for example, SEQ ID NO: 20 and 21 or the amino acid sequences of SEQ ID NO: 22 and 23, for example SEQ ID NO: 24 and 25.

As used herein, the terms “recognizes”, “binds”, “immunospecifically binds,” “immunospecifically recognizes,” “specifically binds,” and “specifically recognizes” are analogous terms, in the context of antibodies and fragments thereof, and refer to molecules that bind to an antigen as such binding is understood by one skilled in the art.

In some embodiments, antigen binding domains that specifically bind to an antigen (e.g., MART-1) bind with a dissociation constant (K_{d}) of about 1×10⁻⁷ M. In some embodiments, the antigen binding domain specifically binds an antigen (e.g., MART-1) with “high affinity” when the K_{d} is about 1×10⁻⁹ M to about 5×10⁻⁹ M. In some embodiments, the antigen binding domain specifically binds an antigen (e.g., MART-1) with “very high affinity” when the K_{d} is 1×10⁻¹° M to about 5×10⁻¹° M.

The terms “VL”, “VL region,” and “VL domain” are used interchangeably to refer to the light chain variable region of an antigen binding domain such as an antibody or an antigen-binding fragment thereof, and comprise one, two, or all three CDRs.

The terms “VH”, “VH region,” and “VH domain” are used interchangeably to refer to the heavy chain variable region of an antigen binding domain such as an antibody or an antigen-binding fragment thereof, and comprise one, two, or all three CDRs.

A number of definitions of CDRs are commonly in use: Kabat numbering, Chothia numbering, contact numbering, AbM numbering, or IMGT numbering. Kabat numbering is the most commonly used, Chothia numbering is based on structure and defines CDRs by loop position, and IMGT numbering most broadly covers CDRs beyond loops in both directions.

The term “Kabat numbering” and like terms are recognized in the art and refer to a system of numbering amino acid residues in the heavy and light chain variable regions of an antibody, or an antigen binding domain thereof. In some aspects, the CDRs of an antibody may be determined according to the Kabat numbering system (see, e.g., Kabat et al. “Sequences of Proteins of immunological Interest”, 5th Ed., NIH Publication 91-3242, Bethesda Md. 1991). Using the Kabat numbering system, CDRs within an antibody heavy chain molecule are typically present at amino acid positions 31 to 35, which optionally may include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

In some embodiments, the CDRs of the antibodies described herein may be described according to the Kabat numbering scheme (although they can readily be construed in other numbering systems). Tables 1 and 2 provide the CDRs for two exemplary MART-1 antigen binding domains using the Kabat numbering scheme:

TABLE 1
CDR Table (Kabat)

| | | SEQ | | SEQ | | SEQ |
|---|---|---|---|---|---|---|
| Sequence | CDR1 | ID NO | CDR2 | ID NO | CDR3 | ID NO |
| M7_VL | SASQGIHNY | 1 | YTSSLHS | 2 | QQYSKLPRT | 3 |
| | LN | | | | | |
| M7_VH | TSGMNVG | residues | HIWWNDDKYY | 8 | SYFGDYVWYFD | 9 |
| | 6-12 | of | NPALKS | | V | |
| | | SEQ ID | | | | |
| | | NO: 7 | | | | |

TABLE 2
CDR Table (Kabat)

| | | SEQ | | SEQ | | SEQ |
|---|---|---|---|---|---|---|
| Sequence | CDR1 | ID NO | CDR2 | ID NO | CDR3 | ID NO |
| M1_VL | QASQSVYKN | 4 | GASTLAS | 5 | AGEYNNMLYP | 6 |
| | NRLS | | | | | |
| M1_VH | SPVMI | 12 | IISISGNTGY | 13 | MGYDSSSGYAW | 14 |
| | | | ASWA | | NL | |

In some aspects, the CDRs of an antibody may be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). When using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

Tables 3 and 4 below provide the CDRs for two exemplary MART-1 antigen binding domains which have been determined according to the Chothia numbering scheme:

TABLE 3
CDR Table (Chothia)

| | | SEQ | | SEQ | | SEQ |
|---|---|---|---|---|---|---|
| Sequence | CDR1 | ID NO | CDR2 | ID NO | CDR3 | ID NO |
| M7_VL | SASQGIHNYLN | 1 | YTSSLHS | 2 | QQYSKLPRT | 3 |
| M7_VH | GFSLNTSGM | 10 | WWNDD | 11 | SYFGDYVWYFD | 9 |
| | | | | | V | |

TABLE 4
CDR Table (Chothia)

| | | SEQ | | SEQ | | SEQ |
|---|---|---|---|---|---|---|
| Sequence | CDR1 | ID NO | CDR2 | ID NO | CDR3 | ID NO |
| M1_VL | QASQSVYKN | 4 | GASTLAS | 5 | AGEYNNMLYP | 6 |
| | NRLS | | | | | |
| M1_VH | GFSISSP | 15 | SIS | 16 | MGYDSSSGYAW | 14 |
| | | | | | NL | |

The IMGT numbering scheme relies on the high conservation of the structure of the variable region across species. This numbering was set up after aligning more than 5,000 sequences. It takes into account and combines the definition of the framework (FR) and complementarity determining regions (CDR), structural data from X-ray diffraction studies, and the characterization of the hypervariable loops. See, e.g., Lefranc, M.-P. et al., Dev. Comp. Immunol., 27, 55-77 (2003).

Tables 5 and 6 below provide the CDRs for two exemplary MART-1 antigen binding domains which have been determined according to the IMGT numbering scheme:

TABLE 5
CDR Table (IMGT)

| | | SEQ | | SEQ | | SEQ |
|---|---|---|---|---|---|---|
| Sequence | CDR1 | ID NO | CDR2 | ID NO | CDR3 | ID NO |
| M1_VL | QASQSVYKNNR | 4 | GASTLAS | 5 | AGEYNNMLYP | 6 |
| | LS | | | | | |
| M1_VH | GFSISSPVMI | 17 | IISISGNTGYAS | 13 | MGYDSSSGYAW | 14 |
| | | | WA | | NL | |

TABLE 6
CDR Table (IMGT)

| | | SEQ | | SEQ | | SEQ |
|---|---|---|---|---|---|---|
| Sequence | CDR1 | ID NO | CDR2 | ID NO | CDR3 | ID NO |
| M7_VL | SASQGIHNYLN | 1 | YTSSLHS | 2 | QQYSKLPRT | 3 |
| M7_VH | GFSLNTSGMNV | 7 | HIWWNDDKYYNP | 8 | SYFGDYVWYFD | 9 |
| | G | | ALKS | | V | |

The CDRs listed in Tables 1 to 6, were identified using the Molecular Operating Environment (see, the World Wide Web (www) at chemcomp.com/MOE-Molecular_Operating_Environment.htm.

As used herein, the term “lymphocyte” means a white blood cell found in a vertebrate's immune system. Lymphocytes include natural killer (NK) cells, T cells and B cells. NK cells are a type of cytotoxic (cell toxic) lymphocyte that represent a major component of the inherent immune system. NK cells reject tumors and cells infected by viruses through the process of apoptosis or programmed cell death. They were termed “natural killers” because they do not require activation in order to kill cells.

T cells play a major role in cell-mediated-immunity (no antibody involvement). Types of T cells include: (1) helper T cells (e.g., CD4+ cells); (2) cytotoxic T cells (also known as TC, cytotoxic T lymphocyte, CTL, T-killer cell, cytolytic T cell, CD8+ T-cells or killer T cell); (3) memory T-cells, including: (i) stem memory T_{SCM} cells which, like naive cells, are CD45RO−, CCR7+, CD45RA+, CD62L+(L-selectin), CD27+, CD28+ and IL-7Ra+, but they also express large amounts of CD95, IL-2Rβ, CXCR3, and LFA-1, and show numerous functional attributes distinctive of memory cells); (ii) central memory T_{CM} cells, which express L-selectin and the CCR7, they secrete IL-2, but not IFNγ or IL-4; and (iii) effector memory T_{EM} cells, however, do not express L-selectin or CCR7 but produce effector cytokines like IFNγ and IL-4); (4) regulatory T-cells (Tregs, suppressor T cells, or CD4+CD25+ regulatory T cells); (5) natural killer T cells (NKTs); (6) γδ (Gamma Delta) T cells; and (7) mucosal associated invariant T cells (MAITs).

As used herein, the term “cytokine” means a non-antibody protein that is released by one cell in response to contact with a specific antigen, wherein the cytokine interacts with a second cell to mediate a response in the second cell. A cytokine can be endogenously expressed by a cell or administered to a subject. Cytokines can be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin 7 (IL-7) and interleukin 15 (IL-15), promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

As used herein, the terms “genetic engineering” or “engineering” are used interchangeably and mean a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR), which is incorporated into the cell's genome.

As used herein, the terms “transduction” and “transduced” means the process whereby foreign DNA is introduced into a cell via viral vector (see Hartl and Jones (1997) Genetics: Principles and Analysis, 4^{th} ed, Jones & Bartlett). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

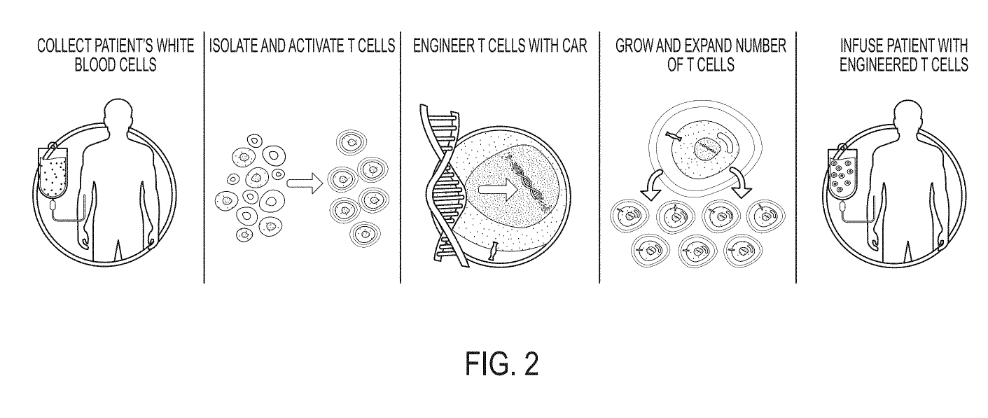

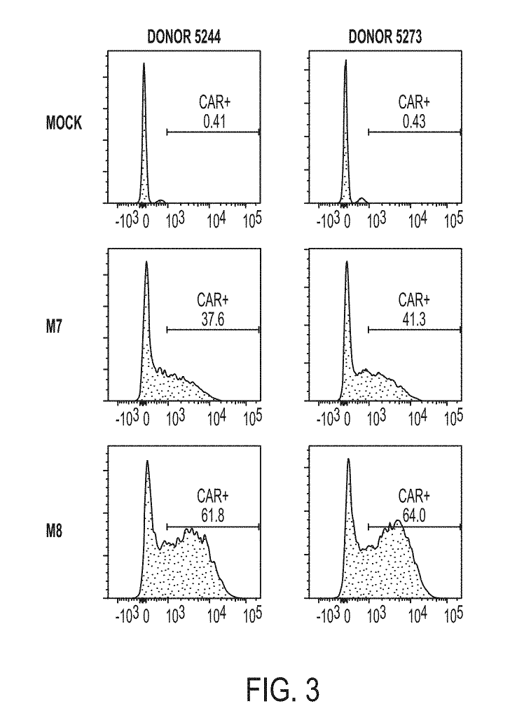

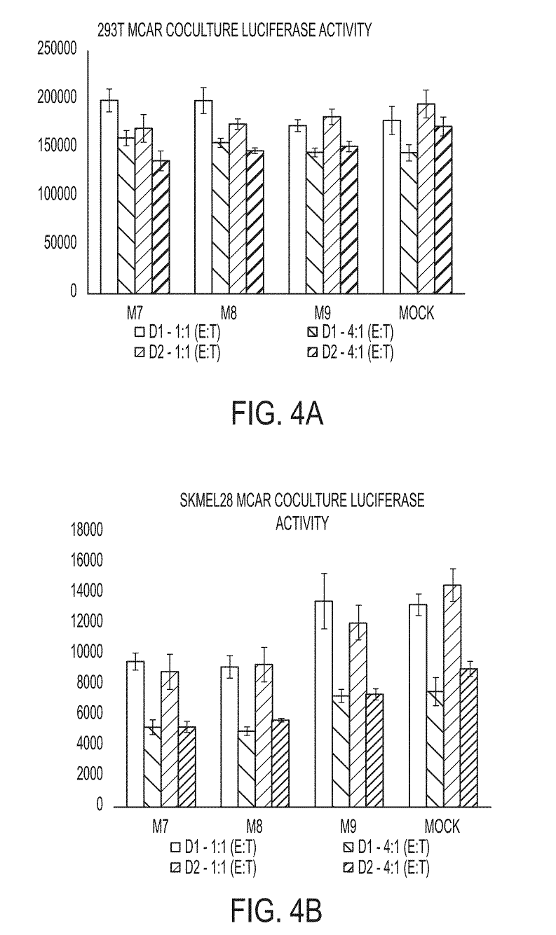

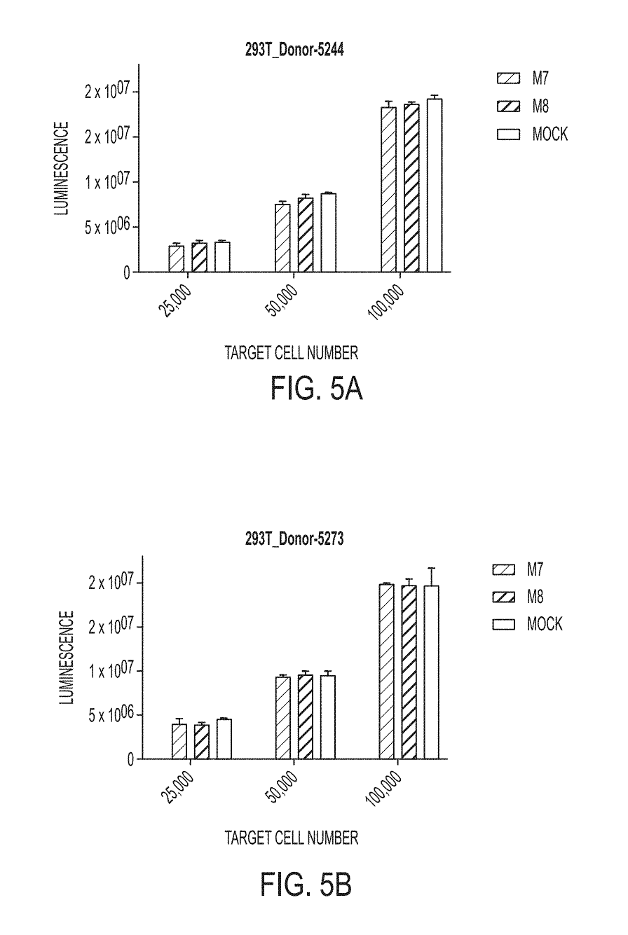

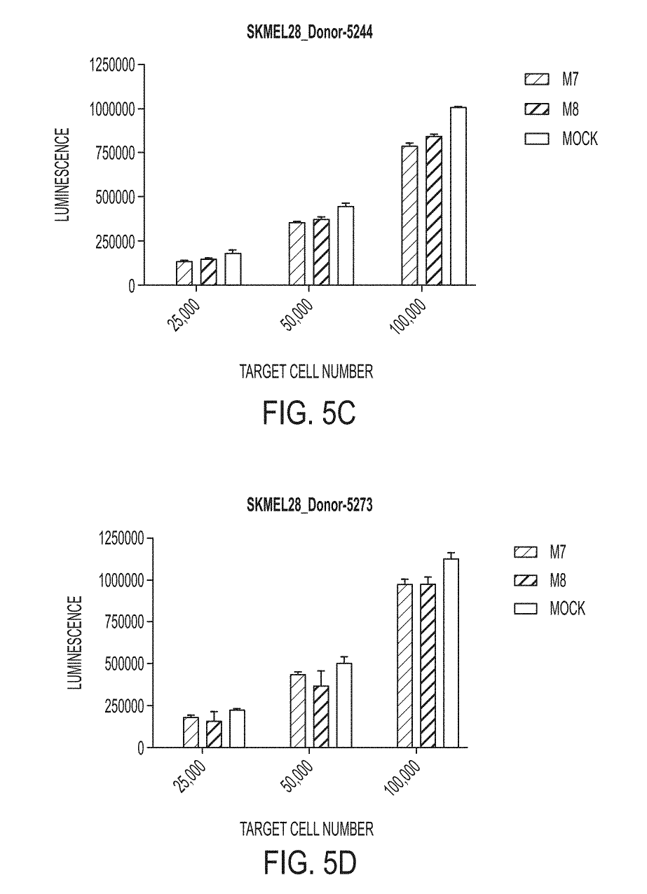

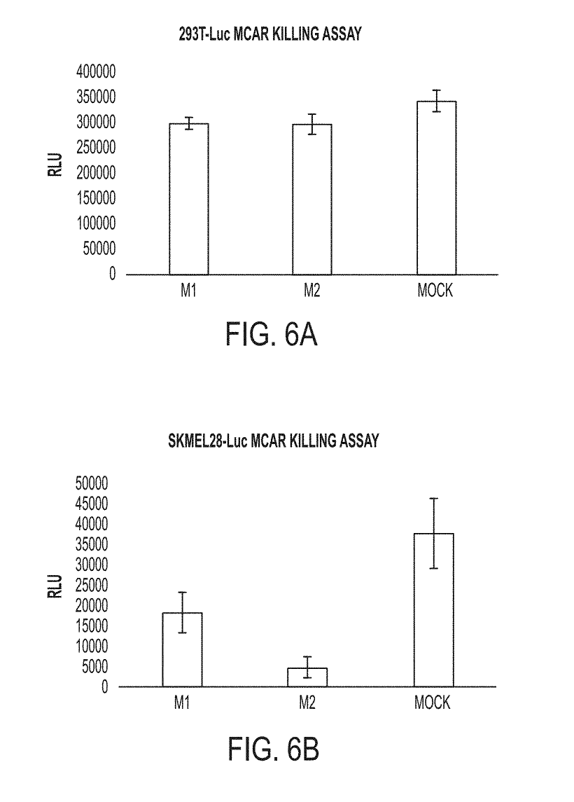

As used herein, the term “autologous” mean any material derived from the same individual to which it is later to be re-introduced. For example, the Engineered Autologous Cell Therapy (eACT™), also known as adoptive cell transfer, is a process by which a patient's own T cells are collected and subsequently genetically engineered to express a polynucleotide, e.g., a polynucleotide encoding a CAR that recognizes and targets one or more antigens expressed on the cell surface of one or more specific tumor cells or malignancies, and then administered back to the same patient. See, FIG. 2 for a brief summary of the steps involved in eACT™.

As used herein the term “allogeneic” means any material derived from one individual which is then introduced to another individual of the same species, e.g., allogeneic T cell transplantation.

“Administering” refers to the physical introduction of an agent to a subject, using any of the various methods and delivery systems known to those skilled in the art. Exemplary routes of administration for the formulations of the present invention include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering may also be performed, for example, once, a plurality of times, and/or over one or more extended periods.

Administering a composition of the present invention or a plurality of cells (which express an engineered CAR) of the present invention will produce an “anti-tumor effect” or an “anti-cancer effect”. As used herein, the term “anti-tumor effect” or “anti-cancer effect” means a biological effect that may present as a decrease in tumor volume, a decrease in the number of tumor or cancer cells, a decrease in tumor cell or cancer cell proliferation, a decrease in the number of metastases, an increase in overall or progression-free survival, an increase in life expectancy, or amelioration of various physiological symptoms associated with the tumor or cancer.

As used herein, the terms “therapeutically effective amount,” “effective dose,” “effective amount,” and “therapeutically effective dosage” of a therapeutic agent, e.g., a composition of the present invention or a plurality of cells (which express an engineered CAR) of the present invention, are used interchangeably and mean any amount that, when used alone or in combination with another therapeutic agent, provides an “anti-tumor effect” or an “anti-cancer effect”.

The ability of a therapeutic agent, as used herein, to provide an “anti-tumor effect” or an “anti-cancer effect” may be evaluated using a variety of methods known to the skilled practitioner, such as in human subjects during clinical trials, in animal model systems predictive of efficacy in humans, or by assaying the activity of the agent in in vitro assays. An “anti-tumor effect” or an “anti-cancer effect” is synonymous with the term “treatment” of a subject and “treating” a subject.

As used herein, the term “immune response” means the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

As used herein, the term “immunotherapy” means the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, Engineered Autologous Cell Therapy (eACT™), and allogeneic T cell transplantation. Those of skill in the art will recognize that the conditioning methods of the present invention would enhance the effectiveness of any transplanted T cell therapy. Examples of T cell therapies are described in U.S. Patent Publication No. 2014/0154228, U.S. Pat. Nos. 5,728,388; 6,406,699; and 8,119,772, International Publication No. WO 2008/081035; Chodon et al, Clinical Cancer Research, 20(9): 2457-65 (2014), and Johnson et al, Blood, 114(2): 535-46 (2009), each of which are incorporated by reference in its entirety.

The T cells of an immunotherapy may come from any source. For example, T cells may be differentiated in vitro from a stem cell population, or T cells may be obtained from a subject. T cells may also be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, T cells may be derived from one or more available T cell lines. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

As used herein, an “epitope” is a term in the art and refers to a localized region of an antigen to which an antigen binding protein, antigen binding domain, scFv or antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antigen binding protein, antigen binding domain, scFv or antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege et al., (1994) Acta Crystallogr D Biol Crystallogr 50(Pt 4): 339-350; McPherson, (1990) Eur J Biochem 189: 1-23; Chayen, (1997) Structure 5: 1269-1274; McPherson, (1976) J Biol Chem 251: 6300-6303). Antibody:antigen crystals can be studied using well known X-ray diffraction techniques and may be refined using computer software such as X-PLOR (Yale University, 1992, distributed by Molecular Simulations, Inc.; see, e.g., Meth Enzymol (1985) Vols 114 & 115, eds Wyckoff et al.), and BUSTER (Bricogne, (1993) Acta Crystallogr D Biol Crystallogr 49(Pt 1): 37-60; Bricogne, (1997) Meth Enzymol 276A: 361-423, ed. Carter; Roversi et al., (2000) Acta Crystallogr D Biol Crystallogr 56(Pt 10): 1316-1323). Mutagenesis mapping studies can be accomplished using any method known to one of skill in the art. See, e.g., Champe et al., (1995) J Biol Chem 270: 1388-94 and Cunningham & Wells, (1989) Science 244: 1081-85 for a description of mutagenesis techniques, including alanine and arginine scanning mutagenesis techniques.

### DETAILED DESCRIPTION

The present invention provides Chimeric Antigen Receptors (CARs) comprising antigen binding domains that specifically bind MART-1 (e.g., an extracellular epitope of MART-1). The present invention further provides polynucleotides encoding such CARs. The present invention also provides vectors (e.g., viral vectors) comprising such polynucleotides. The present invention additionally provides engineered cells (e.g., T cells) comprising such polynucleotides and/or transduced with such viral vectors. The present invention provides compositions (e.g., pharmaceutical compositions) including a plurality of engineered T cells. And, the present invention provides methods for manufacturing such engineered T cells and compositions and uses (e.g., in treating a melanoma) of such engineered T cells and compositions.

### I. Chimeric Antigen Receptors (CARs)

The present invention relates to chimeric antigen receptors (CARs) comprising an antigen binding domain, such as an scFv, that specifically binds to MART-1, e.g., an extracellular epitope of MART-1, and engineered T cells comprising an antigen binding domain that specifically binds to MART-1. In some embodiments, an antigen binding domain of present invention is an scFv derived from an antibody, e.g., the A103 hybridoma and the EP1422Y hybridoma. Other antibodies directed to MART-1, e.g., an extracellular epitope of MART-1, may be used.

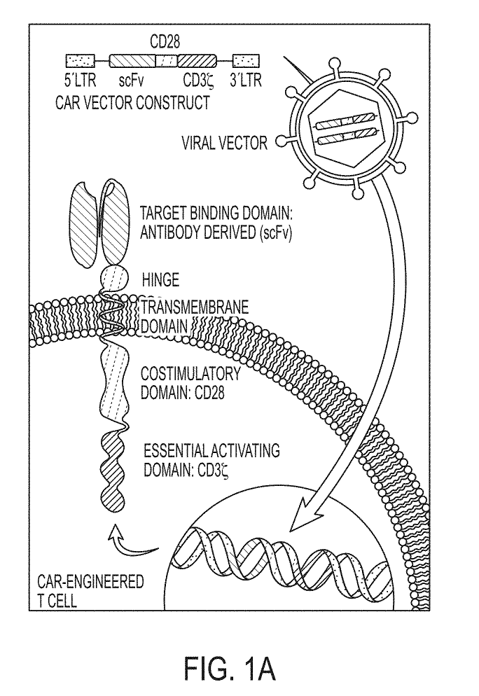

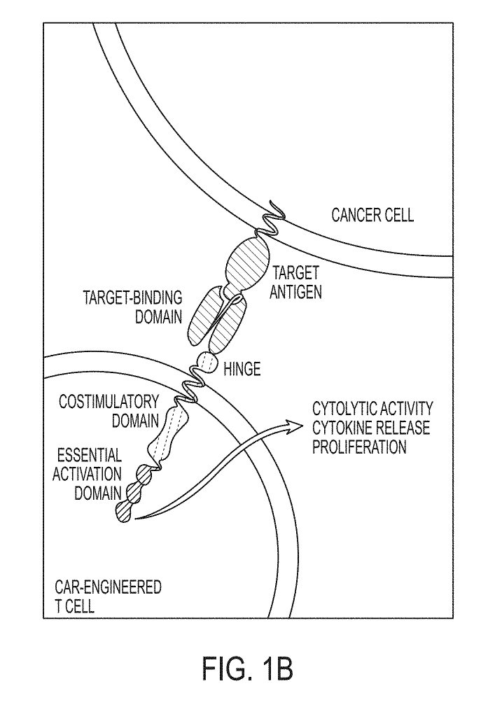

Steps performed in manufacturing a cell that expresses a CAR are shown in FIG. 1A and the steps in which a CAR kills its target cell are shown in FIG. 1B.

An anti-MART-1 CAR of the present invention comprises an antigen binding domain that specifically binds to MART-1. In some embodiments, the anti-MART-1 CAR further comprises a costimulatory domain, and/or an extracellular domain (i.e., a “hinge” or “spacer” region), and/or a transmembrane domain, and/or an intracellular (signaling) domain, and/or a CD3 zeta activating domain. In some embodiments, the anti-MART-1 CAR comprises an scFv antigen binding domain that specifically binds MART-1, a costimulatory domain, an extracellular domain, a transmembrane domain, and a CD3 zeta activating domain.

It will further be appreciated that where desired, the various domains and regions described herein may be expressed in a separate chain from the antigen binding domain (e.g., scFv) and activating domains, in a so-called “trans” configuration. Thus, in one embodiment an activating domain may be expressed on one chain, while the antigen binding domain, and/or an extracellular domain, and/or a transmembrane domain and/or a costimulatory domain (depending on the desired construction of the CAR) may be expressed on a separate chain.

As described more fully herein, it will be further appreciated that the N- to C-terminal, or extracellular to intracellular, order of the components of a CAR of present invention may be varied as desired. The antigen binding domain (the scFv) will be extracellular in order to associate with the target antigen, and may include a leader or signal peptide at the N terminal end the scFv that is most distal to the cell membrane.

An exemplary orientation and ordering for a CAR of the present invention is: optional “signal peptide” or “leader sequence” (e.g., the leader sequence of CD8a)—anti-MART-1 scFv—optional mini-linker, such as GGGGS (SEQ ID NO: 72), GSG or AAA-hinge—optional mini-linker, such as GGGGS (SEQ ID NO: 72), GSG or AAA—transmembrane region (e.g., CD8a transmembrane region)—optional mini-linker, such as GGGGS (SEQ ID NO: 72), GSG or AAA—costimulatory region (e.g., CD28 or a subsequence of 4-1BB)—optional mini-linker, such as GGGGS (SEQ ID NO: 72), GSG or AAA—activation domain (e.g., a CD3 zeta domain, such as one of those provided herein). In some embodiments, the CAR comprises sequential repeats of the short polypeptide mini-linker. In some embodiments, the CAR comprises 2, 3, 4, or 5 sequential repeats of the mini-linker.

Another exemplary orientation and ordering for a CAR of the present invention comprises two costimulatory domains and is: optional leader sequence (e.g., the leader sequence of CD8a)—anti-MART-1 scFv—optional mini-linker, such as GGGGS (SEQ ID NO: 72), GSG, EAAAK (SEQ ID NO: 93) or AAA-hinge—optional mini-linker, such as GGGGS (SEQ ID NO: 72), GSG or AAA—transmembrane region (e.g., CD8a transmembrane region)—optional mini-linker, such as GGGGS (SEQ ID NO: 72), GSG, EAAAK (SEQ ID NO: 93) or AAA—costimulatory region (e.g., CD28 or a subsequence of 4-1BB)—costimulatory region (e.g., CD28 or a subsequence of 4-1BB)—optional mini-linker, such as GGGGS (SEQ ID NO: 72), GSG or AAA—activation domain (e.g., a CD3 zeta domain, such as one of those provided herein). In some embodiments, the CAR comprises sequential repeats of the short polypeptide mini-linker. In some embodiments, the CAR comprises 2, 3, 4, or 5 sequential repeats of the mini-linker.

### II. The M7 and M8 CARs

As mentioned above, the “M7” and “M8” sequences comprise antigen binding domain sequences or fragments thereof obtained from or modified from mouse monoclonal antibodies derived from the “A103” hybridoma. The CDRs for the A103 hybridoma are shown above in Tables 1, 3, and 5. The M7 and M8 CAR amino acid sequences each comprise an antigen binding domain similar to an scFv in that it includes VH and VL domains separated by a linker. In the M7 CAR amino acid sequence, the VH amino acid sequence precedes (is N-terminal) to the VL amino acid sequence. Conversely, in the M8 CAR amino acid sequence, the VL amino acid sequence precedes (is N-terminal) to the VH amino acid sequence.

An antigen binding domain of the present invention comprises one of the following variable (VL and VH) amino acid sequences which is encoded by one of the following variable (VL and VH) DNA sequences; a CAR of the present invention comprises one of the following CAR amino acid sequences which is encoded by one of the following CAR DNA sequences:

A103 hybridoma M7/M8 VL amino acid sequence:
(SEQ ID NO: 18)
DIQMTQTTSSLSASLGDRVTISCSASQGIHNYLNWYQQKPDGTVKLLIYYTSSLHSGVPSRFSG
SGSGTDYSLTISNLEPEDIATYFCQQYSKLPRTFGGGTKLEIKR
A103 hybridoma M7/M8 VH amino acid sequence:
(SEQ ID NO: 19)
QVTLKESGPGILQPSQTLSLTCSFSGFSLNTSGMNVGWIRQPSGKGLDWLAHIWWNDDKYYNPA
LKSRLTISKDTSNNQVFLKIASVVTADTATYYCVRSYFGDYVWYFDVWGAGTTVTVSS
A103 hybridoma M7 CAR amino acid sequence:
(SEQ ID NO: 20)
MALPVTALLLPLALLLHAARPQVTLKESGPGILQPSQTLSLTCSFSGFSLNTSGMNVGWIRQPS
GKGLDWLAHIWWNDDKYYNPALKSRLTISKDTSNNQVFLKIASVVTADTATYYCVRSYFGDYVW
YFDVWGAGTTVTVSSGSTSGSGKPGSGEGSTKGDIQMTQTTSSLSASLGDRVTISCSASQGIHN
YLNWYQQKPDGTVKLLIYYTSSLHSGVPSRFSGSGSGTDYSLTISNLEPEDIATYFCQQYSKLP
RTFGGGTKLEIKRAAALDNEKSNGTIIHVKGKHLCPSPLFPGPSKPFWVLVVVGGVLACYSLLV
TVAFIIFWVRSKRSRLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQKDKMAEAYSEIGMKG
ERRRGKGHDGLYQGLSTATKDTYDALHMQALPPR
A103 hybridoma M8 CAR amino acid sequence:
(SEQ ID NO: 21)
MALPVTALLLPLALLLHAARPDIQMTQTTSSLSASLGDRVTISCSASQGIHNYLNWYQQKPDGT
VKLLIYYTSSLHSGVPSRFSGSGSGTDYSLTISNLEPEDIATYFCQQYSKLPRTFGGGTKLEIK
RGSTSGSGKPGSGEGSTKGQVTLKESGPGILQPSQTLSLTCSFSGFSLNTSGMNVGWIRQPSGK
GLDWLAHIWWNDDKYYNPALKSRLTISKDTSNNQVFLKIASVVTADTATYYCVRSYFGDYVWYF
DVWGAGTTVTVSSAAALDNEKSNGTIIHVKGKHLCPSPLFPGPSKPFWVLVVVGGVLACYSLLV
TVAFIIFWVRSKRSRLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQKDKMAEAYSEIGMKG
ERRRGKGHDGLYQGLSTATKDTYDALHMQALPPR
M7a CAR amino acid sequence:
(SEQ ID NO: 61)
MALPVTALLLPLALLLHAARPQVTLKESGPGILQPSQTLSLTCSFSGFSLNTSGMNVGWIRQPS
GKGLDWLAHIWWNDDKYYNPALKSRLTISKDTSNNQVFLKIASVVTADTATYYCVRSYFGDYVW
YFDVWGAGTTVTVSSGSTSGSGKPGSGEGSTKGDIQMTQTTSSLSASLGDRVTISCSASQGIHN
YLNWYQQKPDGTVKLLIYYTSSLHSGVPSRFSGSGSGTDYSLTISNLEPEDIATYFCQQYSKLP
RTFGGGTKLEIKRGGGGSLDNEKSNGTIIHVKGKHLCPSPLFPGPSKPFWVLVVVGGVLACYSL
LVTVAFIIFWVRSKRSRLLHSDYMNMTPRRPGPTRKHYQPYAPPRDFAAYRSRVKFSRSADAPA
YQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR
M7b CAR amino acid sequence:
(SEQ ID NO: 63)
MALPVTALLLPLALLLHAARPQVTLKESGPGILQPSQTLSLTCSFSGFSLNTSGMNVGWIRQPS
GKGLDWLAHIWWNDDKYYNPALKSRLTISKDTSNNQVFLKIASVVTADTATYYCVRSYFGDYVW
YFDVWGAGTTVTVSSGSTSGSGKPGSGEGSTKGDIQMTQTTSSLSASLGDRVTISCSASQGIHN
YLNWYQQKPDGTVKLLIYYTSSLHSGVPSRFSGSGSGTDYSLTISNLEPEDIATYFCQQYSKLP
RTFGGGTKLEIKRGGGGSGGGGSLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
M7c CAR amino acid sequence:
(SEQ ID NO: 65)
MALPVTALLLPLALLLHAARPQVTLKESGPGILQPSQTLSLTCSFSGFSLNTSGMNVGWIRQPS
GKGLDWLAHIWWNDDKYYNPALKSRLTISKDTSNNQVFLKIASVVTADTATYYCVRSYFGDYVW
YFDVWGAGTTVTVSSGSTSGSGKPGSGEGSTKGDIQMTQTTSSLSASLGDRVTISCSASQGIHN
YLNWYQQKPDGTVKLLIYYTSSLHSGVPSRFSGSGSGTDYSLTISNLEPEDIATYFCQQYSKLP
RTFGGGTKLEIKRGGGGSGGGGSGGGGSLDNEKSNGTIIHVKGKHLCPSPLFPGPSKPFWVLVV
VGGVLACYSLLVTVAFIIFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
M8a CAR amino acid sequence:
(SEQ ID NO: 67)
MALPVTALLLPLALLLHAARPDIQMTQTTSSLSASLGDRVTISCSASQGIHNYLNWYQQKPDGT
VKLLIYYTSSLHSGVPSRFSGSGSGTDYSLTISNLEPEDIATYFCQQYSKLPRTFGGGTKLEIK
RGSTSGSGKPGSGEGSTKGQVTLKESGPGILQPSQTLSLTCSFSGFSLNTSGMNVGWIRQPSGK
GLDWLAHIWWNDDKYYNPALKSRLTISKDTSNNQVFLKIASVVTADTATYYCVRSYFGDYVWYF
DVWGAGTTVTVSSGGGGSLDNEKSNGTIIHVKGKHLCPSPLFPGPSKPFWVLVVVGGVLACYSL
LVTVAFIIFWVRSKRSRLLHSDYMNMTPRRPGPTRKHYQPYAPPRDFAAYRSRVKFSRSADAPA
YQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR
M8b CAR amino acid sequence:
(SEQ ID NO: 69)
MALPVTALLLPLALLLHAARPDIQMTQTTSSLSASLGDRVTISCSASQGIHNYLNWYQQKPDGT
VKLLIYYTSSLHSGVPSRFSGSGSGTDYSLTISNLEPEDIATYFCQQYSKLPRTFGGGTKLEIK
RGSTSGSGKPGSGEGSTKGQVTLKESGPGILQPSQTLSLTCSFSGFSLNTSGMNVGWIRQPSGK
GLDWLAHIWWNDDKYYNPALKSRLTISKDTSNNQVFLKIASVVTADTATYYCVRSYFGDYVWYF
DVWGAGTTVTVSSGGGGSGGGGSLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
M8c CAR amino acid sequence:
(SEQ ID NO: 71)
MALPVTALLLPLALLLHAARPDIQMTQTTSSLSASLGDRVTISCSASQGIHNYLNWYQQKPDGT
VKLLIYYTSSLHSGVPSRFSGSGSGTDYSLTISNLEPEDIATYFCQQYSKLPRTFGGGTKLEIK
RGSTSGSGKPGSGEGSTKGQVTLKESGPGILQPSQTLSLTCSFSGFSLNTSGMNVGWIRQPSGK
GLDWLAHIWWNDDKYYNPALKSRLTISKDTSNNQVFLKIASVVTADTATYYCVRSYFGDYVWYF
DVWGAGTTVTVSSGGGGSGGGGSGGGGSLDNEKSNGTIIHVKGKHLCPSPLFPGPSKPFWVLVV
VGGVLACYSLLVTVAFIIFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
A103 hybridoma M7/M8 VL DNA sequence:
(SEQ ID NO: 26)
GACATACAAATGACCCAGACAACGTCAAGTCTGTCTGCGTCCTTGGGGGACAGGGTCACTATTT
CTTGCTCCGCGAGTCAAGGGATACACAATTATCTTAATTGGTACCAACAGAAGCCGGACGGCAC
TGTCAAATTGTTGATATACTACACCAGCAGCCTTCACTCAGGAGTTCCCTCCCGCTTTAGCGGA
TCCGGATCTGGCACGGATTACAGCCTTACAATCTCTAATCTGGAGCCTGAGGACATTGCAACAT
ATTTTTGCCAGCAATATAGTAAGCTCCCTCGCACGTTCGGCGGAGGTACAAAATTGGAGATAAA
GCGG
A103 hybridoma M7/M8 VH DNA sequence:
(SEQ ID NO: 27)
CAGGTTACCTTGAAGGAAAGCGGTCCTGGTATCCTTCAGCCATCCCAGACTCTCAGCTTGACGT
GCTCTTTTTCCGGATTCTCCTTGAACACGAGCGGTATGAATGTTGGATGGATTAGACAGCCTTC
CGGTAAAGGGCTGGACTGGTTGGCGCACATATGGTGGAATGACGATAAGTATTACAATCCTGCG
CTGAAAAGTAGGTTGACTATATCTAAGGACACATCTAATAACCAGGTATTCCTGAAAATAGCAT
CAGTCGTAACGGCCGATACTGCGACTTATTACTGTGTCCGATCTTATTTTGGGGATTATGTCTG
GTATTTTGATGTTTGGGGAGCTGGGACCACGGTCACAGTGTCAAGC
A103 hybridoma M8 DNA sequence:
(SEQ ID NO: 29)
ATGGCACTCCCCGTAACTGCTCTGCTGCTGCCGTTGGCATTGCTCCTGCACGCCGCACGCCCGG
ACATACAAATGACCCAGACAACGTCAAGTCTGTCTGCGTCCTTGGGGGACAGGGTCACTATTTC
TTGCTCCGCGAGTCAAGGGATACACAATTATCTTAATTGGTACCAACAGAAGCCGGACGGCACT
GTCAAATTGTTGATATACTACACCAGCAGCCTTCACTCAGGAGTTCCCTCCCGCTTTAGCGGAT
CCGGATCTGGCACGGATTACAGCCTTACAATCTCTAATCTGGAGCCTGAGGACATTGCAACATA
TTTTTGCCAGCAATATAGTAAGCTCCCTCGCACGTTCGGCGGAGGTACAAAATTGGAGATAAAG
CGGGGGAGTACGTCCGGCTCAGGTAAACCTGGAAGTGGGGAAGGATCAACGAAAGGCCAGGTTA
CCTTGAAGGAAAGCGGTCCTGGTATCCTTCAGCCATCCCAGACTCTCAGCTTGACGTGCTCTTT
TTCCGGATTCTCCTTGAACACGAGCGGTATGAATGTTGGATGGATTAGACAGCCTTCCGGTAAA
GGGCTGGACTGGTTGGCGCACATATGGTGGAATGACGATAAGTATTACAATCCTGCGCTGAAAA
GTAGGTTGACTATATCTAAGGACACATCTAATAACCAGGTATTCCTGAAAATAGCATCAGTCGT
AACGGCCGATACTGCGACTTATTACTGTGTCCGATCTTATTTTGGGGATTATGTCTGGTATTTT
GATGTTTGGGGAGCTGGGACCACGGTCACAGTGTCAAGCGCCGCTGCCCTTGATAATGAAAAGT
CAAACGGAACAATCATTCACGTGAAGGGCAAGCACCTCTGTCCGTCACCCTTGTTCCCTGGTCC
ATCCAAGCCATTCTGGGTGTTGGTCGTAGTGGGTGGAGTCCTCGCTTGTTACTCTCTGCTCGTC
ACCGTGGCTTTTATAATCTTCTGGGTTAGATCCAAAAGAAGCCGCCTGCTCCATAGCGATTACA
TGAATATGACTCCACGCCGCCCTGGCCCCACAAGGAAACACTACCAGCCTTACGCACCACCTAG
AGATTTCGCTGCCTATCGGAGCAGGGTGAAGTTTTCCAGATCTGCAGATGCACCAGCGTATCAG
CAGGGCCAGAACCAACTGTATAACGAGCTCAACCTGGGACGCAGGGAAGAGTATGACGTTTTGG
ACAAGCGCAGAGGACGGGACCCTGAGATGGGTGGCAAACCAAGACGAAAAAACCCCCAGGAGGG
TCTCTATAATGAGCTGCAGAAGGATAAGATGGCTGAAGCCTATTCTGAAATAGGCATGAAAGGA
GAGCGGAGAAGGGGAAAAGGGCACGACGGTTTGTACCAGGGACTCAGCACTGCTACGAAGGATA
CTTATGACGCTCTCCACATGCAAGCCCTGCCACCTAGG
A103 hybridoma M7 DNA sequence:
(SEQ ID NO: 28)
ATGGCACTCCCCGTAACTGCTCTGCTGCTGCCGTTGGCATTGCTCCTGCACGCCGCACGCCCGC
AGGTTACCTTGAAGGAAAGCGGTCCTGGTATCCTTCAGCCATCCCAGACTCTCAGCTTGACGTG
CTCTTTTTCCGGATTCTCCTTGAACACGAGCGGTATGAATGTTGGATGGATTAGACAGCCTTCC
GGTAAAGGGCTGGACTGGTTGGCGCACATATGGTGGAATGACGATAAGTATTACAATCCTGCGC
TGAAAAGTAGGTTGACTATATCTAAGGACACATCTAATAACCAGGTATTCCTGAAAATAGCATC
AGTCGTAACGGCCGATACTGCGACTTATTACTGTGTCCGATCTTATTTTGGGGATTATGTCTGG
TATTTTGATGTTTGGGGAGCTGGGACCACGGTCACAGTGTCAAGCGGGAGTACGTCCGGCTCAG
GTAAACCTGGAAGTGGGGAAGGATCAACGAAAGGCGACATACAAATGACCCAGACAACGTCAAG
TCTGTCTGCGTCCTTGGGGGACAGGGTCACTATTTCTTGCTCCGCGAGTCAAGGGATACACAAT
TATCTTAATTGGTACCAACAGAAGCCGGACGGCACTGTCAAATTGTTGATATACTACACCAGCA
GCCTTCACTCAGGAGTTCCCTCCCGCTTTAGCGGATCCGGATCTGGCACGGATTACAGCCTTAC
AATCTCTAATCTGGAGCCTGAGGACATTGCAACATATTTTTGCCAGCAATATAGTAAGCTCCCT
CGCACGTTCGGCGGAGGTACAAAATTGGAGATAAAGCGGGCCGCTGCCCTTGATAATGAAAAGT
CAAACGGAACAATCATTCACGTGAAGGGCAAGCACCTCTGTCCGTCACCCTTGTTCCCTGGTCC
ATCCAAGCCATTCTGGGTGTTGGTCGTAGTGGGTGGAGTCCTCGCTTGTTACTCTCTGCTCGTC
ACCGTGGCTTTTATAATCTTCTGGGTTAGATCCAAAAGAAGCCGCCTGCTCCATAGCGATTACA
TGAATATGACTCCACGCCGCCCTGGCCCCACAAGGAAACACTACCAGCCTTACGCACCACCTAG
AGATTTCGCTGCCTATCGGAGCAGGGTGAAGTTTTCCAGATCTGCAGATGCACCAGCGTATCAG
CAGGGCCAGAACCAACTGTATAACGAGCTCAACCTGGGACGCAGGGAAGAGTATGACGTTTTGG
ACAAGCGCAGAGGACGGGACCCTGAGATGGGTGGCAAACCAAGACGAAAAAACCCCCAGGAGGG
TCTCTATAATGAGCTGCAGAAGGATAAGATGGCTGAAGCCTATTCTGAAATAGGCATGAAAGGA
GAGCGGAGAAGGGGAAAAGGGCACGACGGTTTGTACCAGGGACTCAGCACTGCTACGAAGGATA
CTTATGACGCTCTCCACATGCAAGCCCTGCCACCTAGG
M7a DNA sequence:
(SEQ ID NO: 60)
ATGGCACTCCCCGTAACTGCTCTGCTGCTGCCGTTGGCATTGCTCCTGCACGCCGCACGCCCGC
AGGTTACCTTGAAGGAAAGCGGTCCTGGTATCCTTCAGCCATCCCAGACTCTCAGCTTGACGTG
CTCTTTTTCCGGATTCTCCTTGAACACGAGCGGTATGAATGTTGGATGGATTAGACAGCCTTCC
GGTAAAGGGCTGGACTGGTTGGCGCACATATGGTGGAATGACGATAAGTATTACAATCCTGCGC
TGAAAAGTAGGTTGACTATATCTAAGGACACATCTAATAACCAGGTATTCCTGAAAATAGCATC
AGTCGTAACGGCCGATACTGCGACTTATTACTGTGTCCGATCTTATTTTGGGGATTATGTCTGG
TATTTTGATGTTTGGGGAGCTGGGACCACGGTCACAGTGTCAAGCGGGAGTACGTCCGGCTCAG
GTAAACCTGGAAGTGGGGAAGGATCAACGAAAGGCGACATACAAATGACCCAGACAACGTCAAG
TCTGTCTGCGTCCTTGGGGGACAGGGTCACTATTTCTTGCTCCGCGAGTCAAGGGATACACAAT
TATCTTAATTGGTACCAACAGAAGCCGGACGGCACTGTCAAATTGTTGATATACTACACCAGCA
GCCTTCACTCAGGAGTTCCCTCCCGCTTTAGCGGATCCGGATCTGGCACGGATTACAGCCTTAC
AATCTCTAATCTGGAGCCTGAGGACATTGCAACATATTTTTGCCAGCAATATAGTAAGCTCCCT
CGCACGTTCGGCGGAGGTACAAAATTGGAGATAAAGCGGGGAGGGGGTGGAAGTCTTGATAATG
AAAAGTCAAACGGAACAATCATTCACGTGAAGGGCAAGCACCTCTGTCCGTCACCCTTGTTCCC
TGGTCCATCCAAGCCATTCTGGGTGTTGGTCGTAGTGGGTGGAGTCCTCGCTTGTTACTCTCTG
CTCGTCACCGTGGCTTTTATAATCTTCTGGGTTAGATCCAAAAGAAGCCGCCTGCTCCATAGCG
ATTACATGAATATGACTCCACGCCGCCCTGGCCCCACAAGGAAACACTACCAGCCTTACGCACC
ACCTAGAGATTTCGCTGCCTATCGGAGCAGGGTGAAGTTTTCCAGATCTGCAGATGCACCAGCG
TATCAGCAGGGCCAGAACCAACTGTATAACGAGCTCAACCTGGGACGCAGGGAAGAGTATGACG
TTTTGGACAAGCGCAGAGGACGGGACCCTGAGATGGGTGGCAAACCAAGACGAAAAAACCCCCA
GGAGGGTCTCTATAATGAGCTGCAGAAGGATAAGATGGCTGAAGCCTATTCTGAAATAGGCATG
AAAGGAGAGCGGAGAAGGGGAAAAGGGCACGACGGTTTGTACCAGGGACTCAGCACTGCTACGA
AGGATACTTATGACGCTCTCCACATGCAAGCCCTGCCACCTAGGTAA
M7b DNA sequence:
(SEQ ID NO: 62)
ATGGCACTCCCCGTAACTGCTCTGCTGCTGCCGTTGGCATTGCTCCTGCACGCCGCACGCCCGC
AGGTTACCTTGAAGGAAAGCGGTCCTGGTATCCTTCAGCCATCCCAGACTCTCAGCTTGACGTG
CTCTTTTTCCGGATTCTCCTTGAACACGAGCGGTATGAATGTTGGATGGATTAGACAGCCTTCC
GGTAAAGGGCTGGACTGGTTGGCGCACATATGGTGGAATGACGATAAGTATTACAATCCTGCGC
TGAAAAGTAGGTTGACTATATCTAAGGACACATCTAATAACCAGGTATTCCTGAAAATAGCATC
AGTCGTAACGGCCGATACTGCGACTTATTACTGTGTCCGATCTTATTTTGGGGATTATGTCTGG
TATTTTGATGTTTGGGGAGCTGGGACCACGGTCACAGTGTCAAGCGGGAGTACGTCCGGCTCAG
GTAAACCTGGAAGTGGGGAAGGATCAACGAAAGGCGACATACAAATGACCCAGACAACGTCAAG
TCTGTCTGCGTCCTTGGGGGACAGGGTCACTATTTCTTGCTCCGCGAGTCAAGGGATACACAAT
TATCTTAATTGGTACCAACAGAAGCCGGACGGCACTGTCAAATTGTTGATATACTACACCAGCA
GCCTTCACTCAGGAGTTCCCTCCCGCTTTAGCGGATCCGGATCTGGCACGGATTACAGCCTTAC
AATCTCTAATCTGGAGCCTGAGGACATTGCAACATATTTTTGCCAGCAATATAGTAAGCTCCCT
CGCACGTTCGGCGGAGGTACAAAATTGGAGATAAAGCGGGGAGGGGGTGGAAGTGGGGGCGGTG
GCAGCCTTGATAATGAAAAGTCAAACGGAACAATCATTCACGTGAAGGGCAAGCACCTCTGTCC
GTCACCCTTGTTCCCTGGTCCATCCAAGCCATTCTGGGTGTTGGTCGTAGTGGGTGGAGTCCTC
GCTTGTTACTCTCTGCTCGTCACCGTGGCTTTTATAATCTTCTGGGTTAGATCCAAAAGAAGCC
GCCTGCTCCATAGCGATTACATGAATATGACTCCACGCCGCCCTGGCCCCACAAGGAAACACTA
CCAGCCTTACGCACCACCTAGAGATTTCGCTGCCTATCGGAGCAGGGTGAAGTTTTCCAGATCT
GCAGATGCACCAGCGTATCAGCAGGGCCAGAACCAACTGTATAACGAGCTCAACCTGGGACGCA
GGGAAGAGTATGACGTTTTGGACAAGCGCAGAGGACGGGACCCTGAGATGGGTGGCAAACCAAG
ACGAAAAAACCCCCAGGAGGGTCTCTATAATGAGCTGCAGAAGGATAAGATGGCTGAAGCCTAT
TCTGAAATAGGCATGAAAGGAGAGCGGAGAAGGGGAAAAGGGCACGACGGTTTGTACCAGGGAC
TCAGCACTGCTACGAAGGATACTTATGACGCTCTCCACATGCAAGCCCTGCCACCTAGGTAA
M7c DNA sequence:
(SEQ ID NO: 64)
ATGGCACTCCCCGTAACTGCTCTGCTGCTGCCGTTGGCATTGCTCCTGCACGCCGCACGCCCGC
AGGTTACCTTGAAGGAAAGCGGTCCTGGTATCCTTCAGCCATCCCAGACTCTCAGCTTGACGTG
CTCTTTTTCCGGATTCTCCTTGAACACGAGCGGTATGAATGTTGGATGGATTAGACAGCCTTCC
GGTAAAGGGCTGGACTGGTTGGCGCACATATGGTGGAATGACGATAAGTATTACAATCCTGCGC
TGAAAAGTAGGTTGACTATATCTAAGGACACATCTAATAACCAGGTATTCCTGAAAATAGCATC
AGTCGTAACGGCCGATACTGCGACTTATTACTGTGTCCGATCTTATTTTGGGGATTATGTCTGG
TATTTTGATGTTTGGGGAGCTGGGACCACGGTCACAGTGTCAAGCGGGAGTACGTCCGGCTCAG
GTAAACCTGGAAGTGGGGAAGGATCAACGAAAGGCGACATACAAATGACCCAGACAACGTCAAG
TCTGTCTGCGTCCTTGGGGGACAGGGTCACTATTTCTTGCTCCGCGAGTCAAGGGATACACAAT
TATCTTAATTGGTACCAACAGAAGCCGGACGGCACTGTCAAATTGTTGATATACTACACCAGCA
GCCTTCACTCAGGAGTTCCCTCCCGCTTTAGCGGATCCGGATCTGGCACGGATTACAGCCTTAC
AATCTCTAATCTGGAGCCTGAGGACATTGCAACATATTTTTGCCAGCAATATAGTAAGCTCCCT
CGCACGTTCGGCGGAGGTACAAAATTGGAGATAAAGCGGGGAGGGGGTGGAAGTGGGGGCGGTG
GCAGCGGCGGTGGCGGCAGTCTTGATAATGAAAAGTCAAACGGAACAATCATTCACGTGAAGGG
CAAGCACCTCTGTCCGTCACCCTTGTTCCCTGGTCCATCCAAGCCATTCTGGGTGTTGGTCGTA
GTGGGTGGAGTCCTCGCTTGTTACTCTCTGCTCGTCACCGTGGCTTTTATAATCTTCTGGGTTA
GATCCAAAAGAAGCCGCCTGCTCCATAGCGATTACATGAATATGACTCCACGCCGCCCTGGCCC
CACAAGGAAACACTACCAGCCTTACGCACCACCTAGAGATTTCGCTGCCTATCGGAGCAGGGTG
AAGTTTTCCAGATCTGCAGATGCACCAGCGTATCAGCAGGGCCAGAACCAACTGTATAACGAGC
TCAACCTGGGACGCAGGGAAGAGTATGACGTTTTGGACAAGCGCAGAGGACGGGACCCTGAGAT
GGGTGGCAAACCAAGACGAAAAAACCCCCAGGAGGGTCTCTATAATGAGCTGCAGAAGGATAAG
ATGGCTGAAGCCTATTCTGAAATAGGCATGAAAGGAGAGCGGAGAAGGGGAAAAGGGCACGACG
GTTTGTACCAGGGACTCAGCACTGCTACGAAGGATACTTATGACGCTCTCCACATGCAAGCCCT
GCCACCTAGGTAA
M8a DNA sequence:
(SEQ ID NO: 66)
ATGGCACTCCCCGTAACTGCTCTGCTGCTGCCGTTGGCATTGCTCCTGCACGCCGCACGCCCGG
ACATACAAATGACCCAGACAACGTCAAGTCTGTCTGCGTCCTTGGGGGACAGGGTCACTATTTC
TTGCTCCGCGAGTCAAGGGATACACAATTATCTTAATTGGTACCAACAGAAGCCGGACGGCACT
GTCAAATTGTTGATATACTACACCAGCAGCCTTCACTCAGGAGTTCCCTCCCGCTTTAGCGGAT
CCGGATCTGGCACGGATTACAGCCTTACAATCTCTAATCTGGAGCCTGAGGACATTGCAACATA
TTTTTGCCAGCAATATAGTAAGCTCCCTCGCACGTTCGGCGGAGGTACAAAATTGGAGATAAAG
CGGGGGAGTACGTCCGGCTCAGGTAAACCTGGAAGTGGGGAAGGATCAACGAAAGGCCAGGTTA
CCTTGAAGGAAAGCGGTCCTGGTATCCTTCAGCCATCCCAGACTCTCAGCTTGACGTGCTCTTT
TTCCGGATTCTCCTTGAACACGAGCGGTATGAATGTTGGATGGATTAGACAGCCTTCCGGTAAA
GGGCTGGACTGGTTGGCGCACATATGGTGGAATGACGATAAGTATTACAATCCTGCGCTGAAAA
GTAGGTTGACTATATCTAAGGACACATCTAATAACCAGGTATTCCTGAAAATAGCATCAGTCGT
AACGGCCGATACTGCGACTTATTACTGTGTCCGATCTTATTTTGGGGATTATGTCTGGTATTTT
GATGTTTGGGGAGCTGGGACCACGGTCACAGTGTCAAGCGGAGGGGGTGGAAGTCTTGATAATG
AAAAGTCAAACGGAACAATCATTCACGTGAAGGGCAAGCACCTCTGTCCGTCACCCTTGTTCCC
TGGTCCATCCAAGCCATTCTGGGTGTTGGTCGTAGTGGGTGGAGTCCTCGCTTGTTACTCTCTG
CTCGTCACCGTGGCTTTTATAATCTTCTGGGTTAGATCCAAAAGAAGCCGCCTGCTCCATAGCG
ATTACATGAATATGACTCCACGCCGCCCTGGCCCCACAAGGAAACACTACCAGCCTTACGCACC
ACCTAGAGATTTCGCTGCCTATCGGAGCAGGGTGAAGTTTTCCAGATCTGCAGATGCACCAGCG
TATCAGCAGGGCCAGAACCAACTGTATAACGAGCTCAACCTGGGACGCAGGGAAGAGTATGACG
TTTTGGACAAGCGCAGAGGACGGGACCCTGAGATGGGTGGCAAACCAAGACGAAAAAACCCCCA
GGAGGGTCTCTATAATGAGCTGCAGAAGGATAAGATGGCTGAAGCCTATTCTGAAATAGGCATG
AAAGGAGAGCGGAGAAGGGGAAAAGGGCACGACGGTTTGTACCAGGGACTCAGCACTGCTACGA
AGGATACTTATGACGCTCTCCACATGCAAGCCCTGCCACCTAGGTAA
M8b DNA sequence:
(SEQ ID NO: 68)
ATGGCACTCCCCGTAACTGCTCTGCTGCTGCCGTTGGCATTGCTCCTGCACGCCGCACGCCCGG
ACATACAAATGACCCAGACAACGTCAAGTCTGTCTGCGTCCTTGGGGGACAGGGTCACTATTTC
TTGCTCCGCGAGTCAAGGGATACACAATTATCTTAATTGGTACCAACAGAAGCCGGACGGCACT
GTCAAATTGTTGATATACTACACCAGCAGCCTTCACTCAGGAGTTCCCTCCCGCTTTAGCGGAT
CCGGATCTGGCACGGATTACAGCCTTACAATCTCTAATCTGGAGCCTGAGGACATTGCAACATA
TTTTTGCCAGCAATATAGTAAGCTCCCTCGCACGTTCGGCGGAGGTACAAAATTGGAGATAAAG
CGGGGGAGTACGTCCGGCTCAGGTAAACCTGGAAGTGGGGAAGGATCAACGAAAGGCCAGGTTA
CCTTGAAGGAAAGCGGTCCTGGTATCCTTCAGCCATCCCAGACTCTCAGCTTGACGTGCTCTTT
TTCCGGATTCTCCTTGAACACGAGCGGTATGAATGTTGGATGGATTAGACAGCCTTCCGGTAAA
GGGCTGGACTGGTTGGCGCACATATGGTGGAATGACGATAAGTATTACAATCCTGCGCTGAAAA
GTAGGTTGACTATATCTAAGGACACATCTAATAACCAGGTATTCCTGAAAATAGCATCAGTCGT
AACGGCCGATACTGCGACTTATTACTGTGTCCGATCTTATTTTGGGGATTATGTCTGGTATTTT
GATGTTTGGGGAGCTGGGACCACGGTCACAGTGTCAAGCGGAGGGGGTGGAAGTGGGGGCGGTG
GCAGCCTTGATAATGAAAAGTCAAACGGAACAATCATTCACGTGAAGGGCAAGCACCTCTGTCC
GTCACCCTTGTTCCCTGGTCCATCCAAGCCATTCTGGGTGTTGGTCGTAGTGGGTGGAGTCCTC
GCTTGTTACTCTCTGCTCGTCACCGTGGCTTTTATAATCTTCTGGGTTAGATCCAAAAGAAGCC
GCCTGCTCCATAGCGATTACATGAATATGACTCCACGCCGCCCTGGCCCCACAAGGAAACACTA
CCAGCCTTACGCACCACCTAGAGATTTCGCTGCCTATCGGAGCAGGGTGAAGTTTTCCAGATCT
GCAGATGCACCAGCGTATCAGCAGGGCCAGAACCAACTGTATAACGAGCTCAACCTGGGACGCA
GGGAAGAGTATGACGTTTTGGACAAGCGCAGAGGACGGGACCCTGAGATGGGTGGCAAACCAAG
ACGAAAAAACCCCCAGGAGGGTCTCTATAATGAGCTGCAGAAGGATAAGATGGCTGAAGCCTAT
TCTGAAATAGGCATGAAAGGAGAGCGGAGAAGGGGAAAAGGGCACGACGGTTTGTACCAGGGAC
TCAGCACTGCTACGAAGGATACTTATGACGCTCTCCACATGCAAGCCCTGCCACCTAGGTAA
M8c DNA sequence:
(SEQ ID NO: 70)
ATGGCACTCCCCGTAACTGCTCTGCTGCTGCCGTTGGCATTGCTCCTGCACGCCGCACGCCCGG
ACATACAAATGACCCAGACAACGTCAAGTCTGTCTGCGTCCTTGGGGGACAGGGTCACTATTTC
TTGCTCCGCGAGTCAAGGGATACACAATTATCTTAATTGGTACCAACAGAAGCCGGACGGCACT
GTCAAATTGTTGATATACTACACCAGCAGCCTTCACTCAGGAGTTCCCTCCCGCTTTAGCGGAT
CCGGATCTGGCACGGATTACAGCCTTACAATCTCTAATCTGGAGCCTGAGGACATTGCAACATA
TTTTTGCCAGCAATATAGTAAGCTCCCTCGCACGTTCGGCGGAGGTACAAAATTGGAGATAAAG
CGGGGGAGTACGTCCGGCTCAGGTAAACCTGGAAGTGGGGAAGGATCAACGAAAGGCCAGGTTA
CCTTGAAGGAAAGCGGTCCTGGTATCCTTCAGCCATCCCAGACTCTCAGCTTGACGTGCTCTTT
TTCCGGATTCTCCTTGAACACGAGCGGTATGAATGTTGGATGGATTAGACAGCCTTCCGGTAAA
GGGCTGGACTGGTTGGCGCACATATGGTGGAATGACGATAAGTATTACAATCCTGCGCTGAAAA
GTAGGTTGACTATATCTAAGGACACATCTAATAACCAGGTATTCCTGAAAATAGCATCAGTCGT
AACGGCCGATACTGCGACTTATTACTGTGTCCGATCTTATTTTGGGGATTATGTCTGGTATTTT
GATGTTTGGGGAGCTGGGACCACGGTCACAGTGTCAAGCGGAGGGGGTGGAAGTGGGGGCGGTG
GCAGCGGCGGTGGCGGCAGTCTTGATAATGAAAAGTCAAACGGAACAATCATTCACGTGAAGGG
CAAGCACCTCTGTCCGTCACCCTTGTTCCCTGGTCCATCCAAGCCATTCTGGGTGTTGGTCGTA
GTGGGTGGAGTCCTCGCTTGTTACTCTCTGCTCGTCACCGTGGCTTTTATAATCTTCTGGGTTA
GATCCAAAAGAAGCCGCCTGCTCCATAGCGATTACATGAATATGACTCCACGCCGCCCTGGCCC
CACAAGGAAACACTACCAGCCTTACGCACCACCTAGAGATTTCGCTGCCTATCGGAGCAGGGTG
AAGTTTTCCAGATCTGCAGATGCACCAGCGTATCAGCAGGGCCAGAACCAACTGTATAACGAGC
TCAACCTGGGACGCAGGGAAGAGTATGACGTTTTGGACAAGCGCAGAGGACGGGACCCTGAGAT
GGGTGGCAAACCAAGACGAAAAAACCCCCAGGAGGGTCTCTATAATGAGCTGCAGAAGGATAAG
ATGGCTGAAGCCTATTCTGAAATAGGCATGAAAGGAGAGCGGAGAAGGGGAAAAGGGCACGACG
GTTTGTACCAGGGACTCAGCACTGCTACGAAGGATACTTATGACGCTCTCCACATGCAAGCCCT
GCCACCTAGGTAA

In some embodiments an amino acid sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

### III. The M1 and M2 CARs

As mentioned above, the “M1” and “M2” sequences comprise antigen binding domain sequences or fragments thereof obtained from or modified from rabbit monoclonal antibodies derived from the “EP1422Y” hybridoma. The CDRs for the EP1422Y hybridoma are shown above in Tables 2, 4, and 6. The M1 and M2 CAR amino acid sequences each comprise an antigen binding domain similar to an scFv in that it includes VH and VL domains separated by a linker. In the M1 CAR amino acid sequence, the VH amino acid sequence precedes (is N-terminal) to the VL amino acid sequence. Conversely, in the M2 CAR amino acid sequence, the VL amino acid sequence precedes (is N-terminal) to the VH amino acid sequence.

An antigen binding domain of the present invention comprises one of the following variable (VL and VH) amino acid sequences which is encoded by one of the following variable (VL and VH) DNA sequences; a CAR of the present invention comprises one of the following CAR amino acid sequences which is encoded by one of the following CAR DNA sequences:

EP1422Y hybridoma M1/M2 VL amino acid sequence:
(SEQ ID NO: 22)
QIVMTQTPASVSAAVGGTVTINCQASQSVYKNNRLSWFQQKPGQPPKLLIYGASTLASGVPSRF
KGSGSGTQFTLTISDVQCDDAATYYCAGEYNNMLYPFGGGTVVVVKG
EP1422Y hybridoma M1/M2 VH amino acid sequence:
(SEQ ID NO: 23)
QSVEEPGGRLVTPGTPLTLTCTVSGFSISSPVMIWVRQAPEKGLEYIGIISISGNTGYASWAKG
RFTISKTTTTVDLKITSPTTEDTATYFCARMGYDSSSGYAWNLWGPGTLVTVSS
EP1422Y hybridoma M1 CAR amino acid sequence:
(SEQ ID NO: 24)
MALPVTALLLPLALLLHAARPQSVEEPGGRLVTPGTPLTLTCTVSGFSISSPVMIWVRQAPEKG
LEYIGIISISGNTGYASWAKGRFTISKTTTTVDLKITSPTTEDTATYFCARMGYDSSSGYAWNL
WGPGTLVTVSSGSTSGSGKPGSGEGSTKGQIVMTQTPASVSAAVGGTVTINCQASQSVYKNNRL
SWFQQKPGQPPKLLIYGASTLASGVPSRFKGSGSGTQFTLTISDVQCDDAATYYCAGEYNNMLY
PFGGGTVVVVKGAAALDNEKSNGTIIHVKGKHLCPSPLFPGPSKPFWVLVVVGGVLACYSLLVT
VAFIIFWVRSKRSRLLHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
EP1422Y hybridoma M2 CAR amino acid sequence:
(SEQ ID NO: 25)
MALPVTALLLPLALLLHAARPQIVMTQTPASVSAAVGGTVTINCQASQSVYKNNRLSWFQQKPG
QPPKLLIYGASTLASGVPSRFKGSGSGTQFTLTISDVQCDDAATYYCAGEYNNMLYPFGGGTVV
VVKGGSTSGSGKPGSGEGSTKGQSVEEPGGRLVTPGTPLTLTCTVSGFSISSPVMIWVRQAPEK
GLEYIGIISISGNTGYASWAKGRFTISKTTTTVDLKITSPTTEDTATYFCARMGYDSSSGYAWN
LWGPGTLVTVSSAAALDNEKSNGTIIHVKGKHLCPSPLFPGPSKPFWVLVVVGGVLACYSLLVT
VAFIIFWVRSKRSRLLHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
M2a CAR amino acid sequence:
(SEQ ID NO: 55)
MALPVTALLLPLALLLHAARPQIVMTQTPASVSAAVGGTVTINCQASQSVYKNNRLSWFQQKPG
QPPKLLIYGASTLASGVPSRFKGSGSGTQFTLTISDVQCDDAATYYCAGEYNNMLYPFGGGTVV
VVKGGSTSGSGKPGSGEGSTKGQSVEEPGGRLVTPGTPLTLTCTVSGFSISSPVMIWVRQAPEK
GLEYIGIISISGNTGYASWAKGRFTISKTTTTVDLKITSPTTEDTATYFCARMGYDSSSGYAWN
LWGPGTLVTVSSGGGGSLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
M2b CAR amino acid sequence:
(SEQ ID NO: 57)
MALPVTALLLPLALLLHAARPQIVMTQTPASVSAAVGGTVTINCQASQSVYKNNRLSWFQQKPG
QPPKLLIYGASTLASGVPSRFKGSGSGTQFTLTISDVQCDDAATYYCAGEYNNMLYPFGGGTVV
VVKGGSTSGSGKPGSGEGSTKGQSVEEPGGRLVTPGTPLTLTCTVSGFSISSPVMIWVRQAPEK
GLEYIGIISISGNTGYASWAKGRFTISKTTTTVDLKITSPTTEDTATYFCARMGYDSSSGYAWN
LWGPGTLVTVSSGGGGSGGGGSLDNEKSNGTIIHVKGKHLCPSPLFPGPSKPFWVLVVVGGVLA
CYSLLVTVAFIIFWVRSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
M2c CAR amino acid sequence:
(SEQ ID NO: 59)
MALPVTALLLPLALLLHAARPQIVMTQTPASVSAAVGGTVTINCQASQSVYKNNRLSWFQQKPG
QPPKLLIYGASTLASGVPSRFKGSGSGTQFTLTISDVQCDDAATYYCAGEYNNMLYPFGGGTVV
VVKGGSTSGSGKPGSGEGSTKGQSVEEPGGRLVTPGTPLTLTCTVSGFSISSPVMIWVRQAPEK
GLEYIGIISISGNTGYASWAKGRFTISKTTTTVDLKITSPTTEDTATYFCARMGYDSSSGYAWN
LWGPGTLVTVSSGGGGSGGGGSGGGGSLDNEKSNGTIIHVKGKHLCPSPLFPGPSKPFWVLVVV
GGVLACYSLLVTVAFIIFWVRSKRSRLLHSDYMNMTPRRPGPTRKHYQPYAPPRDFAAYRSRVK
FSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
EP1422Y hybridoma M1/M2 VL DNA sequence:
(SEQ ID NO: 30)
CAGATTGTGATGACTCAAACACCCGCCTCTGTTTCCGCCGCCGTTGGCGGCACCGTCACCATTA
ACTGCCAGGCAAGTCAATCCGTTTATAAAAACAACAGACTGAGTTGGTTTCAGCAGAAGCCAGG
ACAGCCACCTAAACTTCTGATTTACGGCGCTTCAACTCTGGCATCCGGGGTCCCCAGCAGATTC
AAGGGCTCTGGCTCCGGGACGCAGTTCACTCTGACTATATCTGATGTCCAGTGCGATGACGCCG
CTACATACTACTGTGCCGGCGAATACAATAATATGCTCTATCCTTTCGGCGGCGGGACAGTGGT
CGTGGTCAAAGGC
EP1422Y hybridoma M1/M2 VH DNA sequence:
(SEQ ID NO: 31)
CAGAGTGTCGAAGAACCTGGTGGGAGGCTGGTGACCCCTGGAACTCCACTGACACTGACGTGTA
CAGTGAGCGGTTTTAGCATTTCTTCCCCTGTCATGATTTGGGTTAGACAGGCGCCCGAAAAGGG
ACTGGAATACATCGGTATAATCAGTATCTCCGGAAATACCGGTTACGCCTCATGGGCGAAGGGT
CGATTTACCATTAGCAAAACAACTACCACCGTAGATCTTAAGATCACAAGCCCCACTACAGAGG
ATACAGCCACTTACTTTTGCGCACGAATGGGCTATGATTCCAGCTCAGGCTATGCATGGAACCT
CTGGGGTCCGGGGACGCTGGTCACCGTGTCCTCA
EP1422Y hybridoma M1 CAR DNA sequence:
(SEQ ID NO: 32)
ATGGCACTCCCCGTAACTGCTCTGCTGCTGCCGTTGGCATTGCTCCTGCACGCCGCACGCCCGC
AGAGTGTCGAAGAACCTGGTGGGAGGCTGGTGACCCCTGGAACTCCACTGACACTGACGTGTAC
AGTGAGCGGTTTTAGCATTTCTTCCCCTGTCATGATTTGGGTTAGACAGGCGCCCGAAAAGGGA
CTGGAATACATCGGTATAATCAGTATCTCCGGAAATACCGGTTACGCCTCATGGGCGAAGGGTC
GATTTACCATTAGCAAAACAACTACCACCGTAGATCTTAAGATCACAAGCCCCACTACAGAGGA
TACAGCCACTTACTTTTGCGCACGAATGGGCTATGATTCCAGCTCAGGCTATGCATGGAACCTC
TGGGGTCCGGGGACGCTGGTCACCGTGTCCTCAGGTTCCACTAGTGGATCTGGTAAACCTGGAT
CAGGTGAAGGCTCAACCAAGGGTCAGATTGTGATGACTCAAACACCCGCCTCTGTTTCCGCCGC
CGTTGGCGGCACCGTCACCATTAACTGCCAGGCAAGTCAATCCGTTTATAAAAACAACAGACTG
AGTTGGTTTCAGCAGAAGCCAGGACAGCCACCTAAACTTCTGATTTACGGCGCTTCAACTCTGG
CATCCGGGGTCCCCAGCAGATTCAAGGGCTCTGGCTCCGGGACGCAGTTCACTCTGACTATATC
TGATGTCCAGTGCGATGACGCCGCTACATACTACTGTGCCGGCGAATACAATAATATGCTCTAT
CCTTTCGGCGGCGGGACAGTGGTCGTGGTCAAAGGCGCCGCTGCTCTTGACAACGAGAAATCTA
ACGGGACCATTATCCATGTGAAAGGAAAGCACCTTTGTCCGTCACCGTTGTTCCCCGGGCCTAG
CAAGCCATTTTGGGTGCTCGTCGTGGTGGGAGGCGTGCTGGCTTGCTACTCATTGTTGGTTACC
GTTGCGTTTATCATCTTCTGGGTCAGATCCAAAAGAAGCCGCCTGCTCCATAGCGATTACATGA
ATATGACTCCACGCCGCCCTGGCCCCACAAGGAAACACTACCAGCCTTACGCACCACCTAGAGA
TTTCGCTGCCTATCGGAGCCGAGTGAAATTTTCTAGATCAGCTGATGCTCCCGCCTATCAGCAG
GGACAGAATCAACTTTACAATGAGCTGAACCTGGGTCGCAGAGAAGAGTACGACGTTTTGGACA
AACGCCGGGGCCGAGATCCTGAGATGGGGGGGAAGCCGAGAAGGAAGAATCCTCAAGAAGGCCT
GTACAACGAGCTTCAAAAAGACAAAATGGCTGAGGCGTACTCTGAGATCGGCATGAAGGGCGAG
CGGAGACGAGGCAAGGGTCACGATGGCTTGTATCAGGGCCTGAGTACAGCCACAAAGGACACCT
ATGACGCCCTCCACATGCAGGCACTGCCCCCACGC
EP1422Y hybridoma M2 CAR DNA sequence:
(SEQ ID NO: 33)
ATGGCACTCCCCGTAACTGCTCTGCTGCTGCCGTTGGCATTGCTCCTGCACGCCGCACGCCCGC
AGATCGTGATGACACAGACCCCCGCATCCGTAAGCGCTGCTGTTGGTGGCACAGTGACTATTAA
CTGCCAGGCGTCTCAATCTGTTTATAAAAACAACCGCCTTAGTTGGTTTCAGCAGAAGCCTGGG
CAGCCACCTAAACTGCTGATTTACGGGGCCAGCACGTTGGCAAGCGGGGTACCATCTCGGTTTA
AAGGCTCCGGTTCAGGGACTCAATTCACCTTGACAATCTCCGATGTGCAGTGCGACGATGCAGC
AACATACTATTGCGCAGGGGAGTATAATAATATGCTGTACCCATTTGGAGGCGGGACTGTGGTG
GTTGTTAAAGGCGGCTCTACCTCCGGGTCCGGAAAGCCTGGATCAGGTGAGGGGAGCACAAAAG
GCCAATCTGTCGAGGAGCCCGGTGGCCGCCTGGTGACTCCCGGGACTCCTCTCACCCTGACTTG
TACCGTCAGCGGCTTCAGCATTAGCTCCCCGGTGATGATTTGGGTGCGGCAGGCACCCGAAAAG
GGCCTGGAATACATCGGGATAATCAGCATTTCTGGCAATACGGGCTACGCCAGTTGGGCCAAAG
GCAGATTTACTATCTCTAAAACCACAACCACAGTTGATTTGAAGATCACCAGTCCTACAACCGA
GGATACAGCCACGTATTTTTGCGCACGCATGGGCTACGACTCTAGCTCTGGTTATGCCTGGAAC
CTGTGGGGACCTGGTACCCTTGTTACAGTCTCTAGTGCTGCAGCGCTCGATAATGAGAAGTCCA
ATGGTACAATCATTCACGTGAAGGGTAAACATCTTTGTCCTTCACCCCTCTTCCCGGGACCTAG
CAAGCCGTTCTGGGTTCTCGTCGTGGTGGGCGGCGTTCTGGCCTGCTATAGCCTGCTCGTTACG
GTAGCGTTCATTATCTTTTGGGTTAGATCCAAAAGAAGCCGCCTGCTCCATAGCGATTACATGA
ATATGACTCCACGCCGCCCTGGCCCCACAAGGAAACACTACCAGCCTTACGCACCACCTAGAGA
TTTCGCTGCCTATCGGAGCCGAGTGAAATTTTCTAGATCAGCTGATGCTCCCGCCTATCAGCAG
GGACAGAATCAACTTTACAATGAGCTGAACCTGGGTCGCAGAGAAGAGTACGACGTTTTGGACA
AACGCCGGGGCCGAGATCCTGAGATGGGGGGGAAGCCGAGAAGGAAGAATCCTCAAGAAGGCCT
GTACAACGAGCTTCAAAAAGACAAAATGGCTGAGGCGTACTCTGAGATCGGCATGAAGGGCGAG
CGGAGACGAGGCAAGGGTCACGATGGCTTGTATCAGGGCCTGAGTACAGCCACAAAGGACACCT
ATGACGCCCTCCACATGCAGGCACTGCCCCCACGC
M2a CAR DNA sequence:
(SEQ ID NO: 54)
ATGGCACTCCCCGTAACTGCTCTGCTGCTGCCGTTGGCATTGCTCCTGCACGCCGCACGCCCGC
AGATCGTGATGACACAGACCCCCGCATCCGTAAGCGCTGCTGTTGGTGGCACAGTGACTATTAA
CTGCCAGGCGTCTCAATCTGTTTATAAAAACAACCGCCTTAGTTGGTTTCAGCAGAAGCCTGGG
CAGCCACCTAAACTGCTGATTTACGGGGCCAGCACGTTGGCAAGCGGGGTACCATCTCGGTTTA
AAGGCTCCGGTTCAGGGACTCAATTCACCTTGACAATCTCCGATGTGCAGTGCGACGATGCAGC
AACATACTATTGCGCAGGGGAGTATAATAATATGCTGTACCCATTTGGAGGCGGGACTGTGGTG
GTTGTTAAAGGCGGCTCTACCTCCGGGTCCGGAAAGCCTGGATCAGGTGAGGGGAGCACAAAAG
GCCAATCTGTCGAGGAGCCCGGTGGCCGCCTGGTGACTCCCGGGACTCCTCTCACCCTGACTTG
TACCGTCAGCGGCTTCAGCATTAGCTCCCCGGTGATGATTTGGGTGCGGCAGGCACCCGAAAAG
GGCCTGGAATACATCGGGATAATCAGCATTTCTGGCAATACGGGCTACGCCAGTTGGGCCAAAG
GCAGATTTACTATCTCTAAAACCACAACCACAGTTGATTTGAAGATCACCAGTCCTACAACCGA
GGATACAGCCACGTATTTTTGCGCACGCATGGGCTACGACTCTAGCTCTGGTTATGCCTGGAAC
CTGTGGGGACCTGGTACCCTTGTTACAGTCTCTAGTGGAGGGGGTGGAAGTCTCGATAATGAGA
AGTCCAATGGTACAATCATTCACGTGAAGGGTAAACATCTTTGTCCTTCACCCCTCTTCCCGGG
ACCTAGCAAGCCGTTCTGGGTTCTCGTCGTGGTGGGCGGCGTTCTGGCCTGCTATAGCCTGCTC
GTTACGGTAGCGTTCATTATCTTTTGGGTTAGATCCAAAAGAAGCCGCCTGCTCCATAGCGATT
ACATGAATATGACTCCACGCCGCCCTGGCCCCACAAGGAAACACTACCAGCCTTACGCACCACC
TAGAGATTTCGCTGCCTATCGGAGCCGAGTGAAATTTTCTAGATCAGCTGATGCTCCCGCCTAT
CAGCAGGGACAGAATCAACTTTACAATGAGCTGAACCTGGGTCGCAGAGAAGAGTACGACGTTT
TGGACAAACGCCGGGGCCGAGATCCTGAGATGGGGGGGAAGCCGAGAAGGAAGAATCCTCAAGA
AGGCCTGTACAACGAGCTTCAAAAAGACAAAATGGCTGAGGCGTACTCTGAGATCGGCATGAAG
GGCGAGCGGAGACGAGGCAAGGGTCACGATGGCTTGTATCAGGGCCTGAGTACAGCCACAAAGG
ACACCTATGACGCCCTCCACATGCAGGCACTGCCCCCACGCTAG
M2b CAR DNA sequence:
(SEQ ID NO: 56)
ATGGCACTCCCCGTAACTGCTCTGCTGCTGCCGTTGGCATTGCTCCTGCACGCCGCACGCCCGC
AGATCGTGATGACACAGACCCCCGCATCCGTAAGCGCTGCTGTTGGTGGCACAGTGACTATTAA
CTGCCAGGCGTCTCAATCTGTTTATAAAAACAACCGCCTTAGTTGGTTTCAGCAGAAGCCTGGG
CAGCCACCTAAACTGCTGATTTACGGGGCCAGCACGTTGGCAAGCGGGGTACCATCTCGGTTTA
AAGGCTCCGGTTCAGGGACTCAATTCACCTTGACAATCTCCGATGTGCAGTGCGACGATGCAGC
AACATACTATTGCGCAGGGGAGTATAATAATATGCTGTACCCATTTGGAGGCGGGACTGTGGTG
GTTGTTAAAGGCGGCTCTACCTCCGGGTCCGGAAAGCCTGGATCAGGTGAGGGGAGCACAAAAG
GCCAATCTGTCGAGGAGCCCGGTGGCCGCCTGGTGACTCCCGGGACTCCTCTCACCCTGACTTG
TACCGTCAGCGGCTTCAGCATTAGCTCCCCGGTGATGATTTGGGTGCGGCAGGCACCCGAAAAG
GGCCTGGAATACATCGGGATAATCAGCATTTCTGGCAATACGGGCTACGCCAGTTGGGCCAAAG
GCAGATTTACTATCTCTAAAACCACAACCACAGTTGATTTGAAGATCACCAGTCCTACAACCGA
GGATACAGCCACGTATTTTTGCGCACGCATGGGCTACGACTCTAGCTCTGGTTATGCCTGGAAC
CTGTGGGGACCTGGTACCCTTGTTACAGTCTCTAGTGGAGGGGGTGGAAGTGGGGGCGGTGGCA
GCCTCGATAATGAGAAGTCCAATGGTACAATCATTCACGTGAAGGGTAAACATCTTTGTCCTTC
ACCCCTCTTCCCGGGACCTAGCAAGCCGTTCTGGGTTCTCGTCGTGGTGGGCGGCGTTCTGGCC
TGCTATAGCCTGCTCGTTACGGTAGCGTTCATTATCTTTTGGGTTAGATCCAAAAGAAGCCGCC
TGCTCCATAGCGATTACATGAATATGACTCCACGCCGCCCTGGCCCCACAAGGAAACACTACCA
GCCTTACGCACCACCTAGAGATTTCGCTGCCTATCGGAGCCGAGTGAAATTTTCTAGATCAGCT
GATGCTCCCGCCTATCAGCAGGGACAGAATCAACTTTACAATGAGCTGAACCTGGGTCGCAGAG
AAGAGTACGACGTTTTGGACAAACGCCGGGGCCGAGATCCTGAGATGGGGGGGAAGCCGAGAAG
GAAGAATCCTCAAGAAGGCCTGTACAACGAGCTTCAAAAAGACAAAATGGCTGAGGCGTACTCT
GAGATCGGCATGAAGGGCGAGCGGAGACGAGGCAAGGGTCACGATGGCTTGTATCAGGGCCTGA
GTACAGCCACAAAGGACACCTATGACGCCCTCCACATGCAGGCACTGCCCCCACGCTAG
M2c CAR DNA sequence:
(SEQ ID NO: 58)
ATGGCACTCCCCGTAACTGCTCTGCTGCTGCCGTTGGCATTGCTCCTGCACGCCGCACGCCCGC
AGATCGTGATGACACAGACCCCCGCATCCGTAAGCGCTGCTGTTGGTGGCACAGTGACTATTAA
CTGCCAGGCGTCTCAATCTGTTTATAAAAACAACCGCCTTAGTTGGTTTCAGCAGAAGCCTGGG
CAGCCACCTAAACTGCTGATTTACGGGGCCAGCACGTTGGCAAGCGGGGTACCATCTCGGTTTA
AAGGCTCCGGTTCAGGGACTCAATTCACCTTGACAATCTCCGATGTGCAGTGCGACGATGCAGC
AACATACTATTGCGCAGGGGAGTATAATAATATGCTGTACCCATTTGGAGGCGGGACTGTGGTG
GTTGTTAAAGGCGGCTCTACCTCCGGGTCCGGAAAGCCTGGATCAGGTGAGGGGAGCACAAAAG
GCCAATCTGTCGAGGAGCCCGGTGGCCGCCTGGTGACTCCCGGGACTCCTCTCACCCTGACTTG
TACCGTCAGCGGCTTCAGCATTAGCTCCCCGGTGATGATTTGGGTGCGGCAGGCACCCGAAAAG
GGCCTGGAATACATCGGGATAATCAGCATTTCTGGCAATACGGGCTACGCCAGTTGGGCCAAAG
GCAGATTTACTATCTCTAAAACCACAACCACAGTTGATTTGAAGATCACCAGTCCTACAACCGA
GGATACAGCCACGTATTTTTGCGCACGCATGGGCTACGACTCTAGCTCTGGTTATGCCTGGAAC
CTGTGGGGACCTGGTACCCTTGTTACAGTCTCTAGTGGAGGGGGTGGAAGTGGGGGCGGTGGCA
GCGGCGGTGGCGGCAGTCTCGATAATGAGAAGTCCAATGGTACAATCATTCACGTGAAGGGTAA
ACATCTTTGTCCTTCACCCCTCTTCCCGGGACCTAGCAAGCCGTTCTGGGTTCTCGTCGTGGTG
GGCGGCGTTCTGGCCTGCTATAGCCTGCTCGTTACGGTAGCGTTCATTATCTTTTGGGTTAGAT
CCAAAAGAAGCCGCCTGCTCCATAGCGATTACATGAATATGACTCCACGCCGCCCTGGCCCCAC
AAGGAAACACTACCAGCCTTACGCACCACCTAGAGATTTCGCTGCCTATCGGAGCCGAGTGAAA
TTTTCTAGATCAGCTGATGCTCCCGCCTATCAGCAGGGACAGAATCAACTTTACAATGAGCTGA
ACCTGGGTCGCAGAGAAGAGTACGACGTTTTGGACAAACGCCGGGGCCGAGATCCTGAGATGGG
GGGGAAGCCGAGAAGGAAGAATCCTCAAGAAGGCCTGTACAACGAGCTTCAAAAAGACAAAATG
GCTGAGGCGTACTCTGAGATCGGCATGAAGGGCGAGCGGAGACGAGGCAAGGGTCACGATGGCT
TGTATCAGGGCCTGAGTACAGCCACAAAGGACACCTATGACGCCCTCCACATGCAGGCACTGCC
CCCACGCTAG

In some embodiments an amino acid sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

### IV. CAR Common Element Sequences and Variants

a) Linker Peptide

A CAR comprises an antigen binding domain (e.g., an scFv) including VH and VL domains separated by a linker domain, e.g., of ten to about 25 amino acids. An exemplary linker domain has the following amino acid and DNA sequences:

Linker Peptide amino acid sequence:
(SEQ ID NO: 37)
GSTSGSGKPGSGEGSTKG
Linker Peptide DNA sequence::
(SEQ ID NO: 36)
GGGAGTACGTCCGGCTCAGGTAAACCTGGAAGTGGGGAAGGATCAACGAA
AGGC

Additional linker sequences are provided in the Table of Representative Linkers provided herein above.

In some embodiments an amino acid sequence may be at least about 85%, at least about 90%, at least about 95%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

b) Signal Peptides

In some embodiments, a polynucleotide of the present invention encodes a CAR, wherein the CAR comprises an antigen binding domain that specifically binds to MART-1, and wherein the CAR further comprises a signal peptide (also referred to herein as a “leader sequence” or “signal sequence”). The inclusion of a signal peptide in a CAR of the present invention is optional. If a signal peptide is included in a CAR, it may be expressed on the N terminus of the CAR. Thus, a signal peptide may be contiguous with the VH or VL domain of an antigen binding domain of a CAR, depending on which variable domain is at the N terminal to the other variable domain.

If it is desired to include a signal peptide, such a signal peptide may be synthesized or it may be derived from a naturally occurring molecule. For example, the naturally occurring 21 residue signal peptide of CD8 (see, e.g., Littman et al., (1985) Cell 40:237-46) may be employed as a signal peptide in the CAR polynucleotides of the present invention.

An exemplary “signal peptide” or “leader sequence” has the following amino acid and DNA sequences:

Signal peptide amino acid sequence:
(SEQ ID NO: 35)
MALPVTALLLPLALLLHAARP
Signal peptide DNA sequence:
(SEQ ID NO: 34)
ATGGCACTCCCCGTAACTGCTCTGCTGCTGCCGTTGGCATTGCTCCTGCA
CGCCGCACGCCCG

In some embodiments an amino acid sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

c) Extracellular or Hinge Domains

In some embodiments, a CAR of the present invention comprises an “extracellular domain”, “hinge domain”, “spacer domain”, or “spacer region”, which terms are used interchangeably herein. Such domain may be from or derived from (e.g., comprises all or a fragment of) CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof. A hinge domain may be derived either from a natural or from a synthetic source.

In some embodiments, a hinge domain is positioned between an antigen binding domain (e.g., an scFv) and a transmembrane domain. In this orientation the hinge domain provides distance between the antigen binding domain and the surface of a cell membrane through which the CAR is expressed. In some embodiments, a hinge domain is from or derived from an immunoglobulin. In some embodiments, a hinge domain is selected from the hinge regions of IgG1, IgG2, IgG3, IgG4, IgA, IgD, IgE, and IgM, or a fragment thereof. In other embodiments, a hinge domain comprises, is from, or is derived from the hinge region of CD8 alpha. In some embodiments, a hinge domain comprises, is from, or is derived from the hinge region of CD28. In some embodiments, a hinge domain comprises a fragment of the hinge region of CD8 alpha or a fragment of the hinge region of CD28, wherein the fragment is anything less than the whole hinge region. In some embodiments, the fragment of the CD8 alpha hinge region or the fragment of the CD28 hinge region comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of the CD8 alpha hinge region, or of the CD28 hinge region.

Exemplary hinge domains have the following amino acid and DNA sequences:

CD28 Hinge Domain (variant #1) amino acid sequence,
which also comprises the CD28 TM (underlined) and
intracellular region (bold):
(SEQ ID NO: 39)
LDNEKSNGTIIHVKGKHLCPSPLFPGPSKPFWVLVVVGGVLACYSLLVTV
AFIIFWVRSKRSRLLHSDYMNMTPRRPGPTRKHYQPYAPPRDFAAYRS
CD28 Hinge Domain (variant #1) DNA sequence:
(SEQ ID NO: 38)
CTTGATAATGAAAAGTCAAACGGAACAATCATTCACGTGAAGGGCAAGCA
CCTCTGTCCGTCACCCTTGTTCCCTGGTCCATCCAAGCCATTCTGGGTGT
TGGTCGTAGTGGGTGGAGTCCTCGCTTGTTACTCTCTGCTCGTCACCGTG
GCTTTTATAATCTTCTGGGTTAGATCCAAAAGAAGCCGCCTGCTCCATAG
CGATTACATGAATATGACTCCACGCCGCCCTGGCCCCACAAGGAAACACT
ACCAGCCTTACGCACCACCTAGAGATTTCGCTGCCTATCGGAGC
CD28 Hinge Domain (variant #2) amino acid sequence:
(SEQ ID NO: 51)
IEVMYPPPYLDNEKSNGTIIHVKGKHLCPSPLFPGPSKP
CD28 Hinge Domain (variant #2) DNA sequence:
(SEQ ID NO: 50)
ATTGAGGTGATGTATCCACCGCCTTACCTGGATAACGAAAAGAGTAACGGT
ACCATCATTCACGTGAAAGGTAAACACCTGTGTCCTTCTCCCCTCTTCCCC
GGGCCATCAAAGCCC
CD28 Hinge Domain (Extracellular) amino acid
sequence:
(SEQ ID NO: 41)
LDNEKSNGTIIHVKGKHLCPSPLFPGPSKP
CD28 Hinge Domain (Extracellular) DNA sequence:
(SEQ ID NO: 40)
CTTGATAATGAAAAGTCAAACGGAACAATCATTCACGTGAAGGGCAAGC
ACCTCTGTCCGTCACCCTTGTTCCCTGGTCCATCCAAGCCA

In some embodiments an amino acid sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

The above-mentioned CD28 Hinge Domain variant #1 represents a single sequence comprising at least hinge and transmembrane domains or hinge, transmembrane, and signaling domains (as described further below).

Optionally, a CAR may further comprise a short peptide or polypeptide linker, e.g., between two and ten amino acids in length, which forms a linkage between the hinge domain and the antigen binding domain, or between the hinge domain and a transmembrane domain of a CAR. In examples, a glycine-serine doublet (GS), glycine-serine-glycine triplet (GSG), alanine-alanine-alanine triplet (AAA), EAAAK (SEQ ID NO: 93) or G4S peptide (GGGGS) (SEQ ID NO: 72) provides a suitable linker. In some embodiments, the CAR comprises sequential repeats of the short polypeptide linker. In some embodiments, the CAR comprises 2, 3, 4, or 5 sequential repeats of the linker. The Table of Representative Linkers provided above shows other possible linkers that can be used to join VH and VL domains.

d) Transmembrane (TM) Domains

A CAR of the present invention may further comprise a transmembrane (TM) domain. A transmembrane domain may be designed to be fused to the hinge domain. It may similarly be fused to an intracellular domain, such as a costimulatory domain. In some embodiment, a transmembrane domain that naturally is associated with one of the domains in a CAR may be used. For example, a transmembrane domain may comprise the natural transmembrane region of a costimulatory domain (e.g., the TM region of a CD28 or 4-1BB employed as a costimulatory domain) or the natural transmembrane domain of a hinge region (e.g., the TM region of a CD8 alpha or CD28 employed as a hinge domain).

In some embodiments, the transmembrane domain may be selected or modified by amino acid substitution to avoid binding of such domains to the transmembrane domains of the same or different surface membrane proteins to minimize interactions with other members of the receptor complex. A transmembrane domain may be derived either from a natural or from a synthetic source. When the transmembrane domain is derived from a naturally-occurring source, the domain may be derived from any membrane-bound or transmembrane protein. In some embodiments, a transmembrane domain is derived from CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and combinations thereof.

In some embodiments, a transmembrane domain may comprise a sequence that spans a cell membrane, but extends into the cytoplasm of a cell and/or into the extracellular space. For example, a transmembrane may comprise a membrane-spanning sequence which itself may further comprise 1, 2, 3, 4, 5, 6, 7, 8, 9, 10 or more amino acids that extend into the cytoplasm of a cell, and/or the extracellular space. Thus, a transmembrane domain may comprise a membrane-spanning region, yet may further comprise an amino acid(s) that extend beyond the internal or external surface of the membrane itself; such sequences may still be considered to be a “transmembrane domain.”

The transmembrane (TM) domain may be distinct from the hinge domain (e.g., as described above) or the hinge and TM domains may be comprise a single domain, i.e., a hinge/TM domain. An exemplary TM domain and an exemplary hinge/TM domain have the following amino acid and DNA sequences:

CD28 TM Domain amino acid sequence:
(SEQ ID NO: 43)
FWVLVVVGGVLACYSLLVTVAFIIFWV
CD28 TM Domain DNA sequences:
(SEQ ID NO: 42)
TTCTGGGTGTTGGTCGTAGTGGGTGGAGTCCTCGCTTGTTACTCTCTG
CTCGTCACCGTGGCTTTTATAATCTTCTGGGTT
CD8 Hinge/TM Domain amino acid sequence:
(SEQ ID NO: 53)
AAALSNSIMYFSHFVPVFLPAKPTTTPAPRPPTPAPTIASQPLSLRPE
ACRPAAGGAVHTRGLDFACDIYIWAPLAGTCGVLLLSLVITLYCNHRN
CD8 Hinge/TM Domain DNA sequence:
(SEQ ID NO: 52)
GCTGCAGCATTGAGCAACTCAATAATGTATTTTAGTCACTTTGTACCA
GTGTTCTTGCCGGCTAAGCCTACTACCACACCCGCTCCACGGCCACCT
ACCCCAGCTCCTACCATCGCTTCACAGCCTCTGTCCCTGCGCCCAGAG
GCTTGCCGACCGGCCGCAGGGGGCGCTGTTCATACCAGAGGACTGGAT
TTCGCCTGCGATATCTATATCTGGGCACCCCTGGCCGGAACCTGCGGC
GTACTCCTGCTGTCCCTGGTCATCACGCTCTATTGTAATCACAGGAAC

In some embodiments an amino acid sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

Optionally, a CAR may further comprise a short peptide or polypeptide linker, e.g., between two and ten amino acids in length, which forms a linkage between the transmembrane domain and a proximal cytoplasmic signaling domain of the CAR, such as a costimulatory or activation domain, or to an antigen binding domain (e.g., an anti-MART-1 scFv). In examples, a glycine-serine doublet (GS), glycine-serine-glycine triplet (GSG), alanine-alanine-alanine triplet (AAA), or G4S peptide (GGGGS) (SEQ ID NO: 72) provides a suitable linker. In some embodiments, the CAR comprises sequential repeats of the short polypeptide linker. In some embodiments, the CAR comprises a 2, 3, 4, or 5 sequential repeats of the linker.

e) Costimulatory or Signaling Domains

In some embodiments, the present invention comprises a CAR, which further comprises a costimulatory domain (also known as a “signaling domain”). In some embodiments, a costimulatory domain is positioned between an antigen binding domain (e.g., an scFv), and an activating domain. In some embodiments, a costimulatory domain may comprise an extracellular domain, and/or a transmembrane domain, in addition to an intracellular signaling domain. In some embodiments, a costimulatory domain may comprise a transmembrane domain and an intracellular signaling domain. In some embodiments, a costimulatory domain may comprise an extracellular domain and a transmembrane domain. In some embodiments a costimulatory domain may comprise an intracellular signaling domain. A CAR or engineered T cell of the present invention may comprise one, two or three costimulatory domains, which may be configured in series or flanking one or more other components of the CAR.

A costimulatory domain of the CARs and engineered T cells of the present invention may provide signaling to an activating domain, which then activates at least one of the normal effector functions of the immune cell. Effector function of a T cell, for example, may be cytolytic activity or helper activity including the secretion of cytokines.

In some embodiments, suitable costimulatory domains include (i.e., comprise), but are not limited to CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

An exemplary costimulatory domain (also known as a signaling domain) has the following amino acid and DNA sequences:

CD28 Signaling Domain (Intracellular)
(SEQ ID NO: 45)
RSKRSRLLHSDYMNMTPRRPGPTRKHYQPYAPPRDFAAYRS
CD28 Signaling Domain DNA (Intracellular)
(SEQ ID NO: 44)
AGATCCAAAAGAAGCCGCCTGCTCCATAGCGATTACATGAATATGACTC
CACGCCGCCCTGGCCCCACAAGGAAACACTACCAGCCTTACGCACCACC
TAGAGATTTCGCTGCCTATCGGAGC

In some embodiments an amino acid sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

A costimulatory signaling sequence of a CAR of the present invention may be directly linked to another costimulatory domain, to an activating domain, to a transmembrane domain, or other component of the CAR in a random or specified order.

Optionally, a CAR may further comprise a short peptide or polypeptide linker, e.g., between two and ten amino acids in length. In examples, a glycine-serine doublet (GS), glycine-serine-glycine triplet (GSG), alanine-alanine-alanine triplet (AAA), EAAAK (SEQ ID NO: 93) or G4S peptide (GGGGS) (SEQ ID NO: 72) provides a suitable linker. In some embodiments, the CAR comprises sequential repeats of the short polypeptide linker. In some embodiments, the CAR comprises 2, 3, 4, or 5 sequential repeats of the linker. The Table of Representative Linkers provided above shows other possible linkers that can be used to join VH and VL domains.

It is further noted that multiple costimulatory domains may be incorporated into a CAR of the present invention. For example, a CD28 costimulatory domain and a 4-1BB costimulatory domain may both be incorporated into a CAR of the present invention and, by virtue of the antigen binding component of the CAR, still be directed against MART-1 and cells expressing MART-1 on their surfaces.

f) Activating Domains

In some embodiments, intracellular domains for use in CARs and/or engineered T cell of the present invention include cytoplasmic sequences of the T cell receptor (TCR) and co-receptors that act in concert to initiate signal transduction following antigen/receptor engagement, as well as any derivative or variant of these sequences and any synthetic sequence that has the same functional capability. CD3 is an element of the T cell receptor on native T cells, and has been shown to be an important intracellular activating element in CARs.

Exemplary activation domains have the following amino acid and DNA sequences:

CD3z Activation Domain (variant #1)
(SEQ ID NO: 47)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
CD3z Activation Domain DNA (variant #1)
(SEQ ID NO: 46)
AGGGTGAAGTTTTCCAGATCTGCAGATGCACCAGCGTATCAGCAGGGCC
AGAACCAACTGTATAACGAGCTCAACCTGGGACGCAGGGAAGAGTATGA
CGTTTTGGACAAGCGCAGAGGACGGGACCCTGAGATGGGTGGCAAACCA
AGACGAAAAAACCCCCAGGAGGGTCTCTATAATGAGCTGCAGAAGGATA
AGATGGCTGAAGCCTATTCTGAAATAGGCATGAAAGGAGAGCGGAGAAG
GGGAAAAGGGCACGACGGTTTGTACCAGGGACTCAGCACTGCTACGAAG
GATACTTATGACGCTCTCCACATGCAAGCCCTGCCACCTAGG
CD3z Activation Domain (variant #2)
(SEQ ID NO: 49)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
CD3z Activation Domain DNA (variant #2)
(SEQ ID NO: 48)
AGGGTGAAGTTTTCCAGATCTGCAGATGCACCAGCGTATAAGCAGGGCC
AGAACCAACTGTATAACGAGCTCAACCTGGGACGCAGGGAAGAGTATGA
CGTTTTGGACAAGCGCAGAGGACGGGACCCTGAGATGGGTGGCAAACCA
AGACGAAAAAACCCCCAGGAGGGTCTCTATAATGAGCTGCAGAAGGATA
AGATGGCTGAAGCCTATTCTGAAATAGGCATGAAAGGAGAGCGGAGAAG
GGGAAAAGGGCACGACGGTTTGTACCAGGGACTCAGCACTGCTACGAAG
GATACTTATGACGCTCTCCACATGCAAGCCCTGCCACCTAGG

In some embodiments an amino acid sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned amino acid sequence. In some embodiments a DNA sequence may be at least about 70%, at least about 75%, at least about 80%, at least about 85%, at least about 90%, at least about 95%, at least about 96%, at least about 97%, at least about 98%, at least about 99%, or about 100% identical to an above-mentioned DNA sequence.

In some embodiments, the polynucleotide of the present invention encodes a CAR, wherein the CAR comprises a signal peptide (P), an antigen binding domain, such as an scFv, that associates with human MART-1 (B), a hinge domain (H), a transmembrane domain (T), one or more costimulatory regions (C), and an activation domain (A), wherein the CAR is configured according to the following: P B H T C A.

In some embodiments the components of the CAR are optionally joined though a linker sequence, such as AAA, GSG, or GGGGS (SEQ ID NO: 72). In some embodiments, the antigen binding domain comprises a VH and a VL, wherein the CAR is configured according to the following: P-VH-VL-H-T-C-A or P-VL-VH-H-T-C-A. In some embodiments, the VH and the VL are connected by a linker (L), wherein the CAR is configured according to the following, from N-terminus to C-terminus: P-VH-L-VL-H-T-C-A or P-VH-L-VL-H-T-C-A. In some embodiments, the CAR comprises sequential repeats of the short polypeptide linker. In some embodiments, the CAR comprises 2, 3, 4, or 5 sequential repeats of the linker.

In some embodiments, a CAR may further comprise a means for indicating the binding of the antigen binding domain with MART-1, if present. The means may be attached to the CAR or incorporated into the amino acid sequence itself. Various means for indicating the presence of an antigen may be used. For example, fluorophores, other molecular probes, or enzymes may be linked to the antigen binding domain and the presence of the antigen binding domain may be observed in a variety of ways. Examples of fluorophores include fluorescein, rhodamine, tetramethylrhodamine, eosin, erythrosin, coumarin, methyl-coumarins, pyrene, Malachite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cascade Yellow and R-phycoerythrin (PE) (Molecular Probes), FITC, Rhodamine, and Texas Red (Pierce), Cy5, Cy5.5, and Cy7 (Amersham Life Science).

### V. Vectors, Cells, and Compositions

Aspects of the present invention include vectors (e.g., viral vectors) comprising a polynucleotide of the present invention. In some embodiments, the present invention is directed to a vector or a set of vectors comprising a polynucleotide encoding a CAR, as described herein. In some embodiments, the present invention is directed to a vector or a set of vectors comprising a polynucleotide encoding a CAR comprising an antigen binding domain that specifically binds to MART-1, e.g., an extracellular epitope of MART-1.

Any vector known in the art may be suitable for the present invention. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. In some embodiments of the present invention one, two or more vectors may be employed. For example, in one embodiment, one or more components of a CAR may be disposed on one vector, while one or more different components of a CAR may be disposed on a different vector.

“Lentivirus” refers to a genus of retroviruses that are capable of infecting dividing and non-dividing cells. Several examples of lentiviruses include the human immunodeficiency virus (HIV); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates.

A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” (Alan J. Cann, Ed., Oxford University Press, (2000)); O Narayan and Clements. 1989. J. Gen. Virology 70:1617-1639 (1989); Fields et al. “Fundamental Virology” Raven Press. (1990); Miyoshi H, Blomer U, Takahashi M, Gage F H, Verma I M. 1998. J. Virol. 72(10):8150-7; and U.S. Pat. No. 6,013,516.

Aspects of the present invention include cells comprising a polynucleotide or a vector of the present invention. In some embodiments, the present invention is directed to host cells, such as in vitro cells, comprising a polynucleotide encoding a CAR, as described herein. In some embodiments, the present invention is directed to host cells, e.g., in vitro cells, comprising a polynucleotide encoding a CAR comprising an antigen binding domain that specifically binds to MART-1, e.g., an extracellular epitope of MART-1.

Any cell may be used as a host cell for the polynucleotides, the vectors, or the polypeptides of the present invention. In some embodiments, the cell may be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobacteriaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some embodiments, the host cell is a human cell. In some embodiments, the cell is an immune cell. In some embodiments, the immune cell is selected from the group consisting of a T cell, a B cell, a tumor infiltrating lymphocyte (TIL), a TCR expressing cell, a natural killer (NK) cell, a dendritic cell, a granulocyte, an innate lymphoid cell, a megakaryocyte, a monocyte, a macrophage, a platelet, a thymocyte, and a myeloid cell. In one embodiment, the immune cell is a T cell. In another embodiment, the immune cell is an NK cell. In some embodiments, the T cell is a tumor-infiltrating lymphocyte (TIL), autologous T cell, an Engineered Autologous T Cell (eACT™), an allogeneic T cell, a heterologous T cell, or any combination thereof.

A cell of the present invention may be obtained through any source known in the art. For example, T cells may be differentiated in vitro from a hematopoietic stem cell population, or T cells may be obtained from a subject. T cells may be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. In some embodiments, the cells collected by apheresis are washed to remove the plasma fraction, and placed in an appropriate buffer or media for subsequent processing. In some embodiments, the cells are washed with PBS. As will be appreciated, a washing step may be used, such as by using a semiautomated flowthrough centrifuge, e.g., the COBE™ 2991 cell processor, the Baxter CYTOMATE™, or the like. In some embodiments, the washed cells are resuspended in one or more biocompatible buffers, or other saline solution with or without buffer. In some embodiments, the undesired components of the apheresis sample are removed. Additional methods of isolating T cells for a T cell therapy are disclosed in U.S. Patent Publication No. 2013/0287748, which is herein incorporated by references in its entirety.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD28+, CD4+, CD8+, CD45RA+, and CD45RO+ T cells may be further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD14, CD20, CD11b, CD16, HLA-DR, and CD8. In some embodiments, flow cytometry and cell sorting are used to isolate cell populations of interest for use in the present invention. Using these standard techniques, the engineered T cells administered to a patient when performing the methods provided herein may comprise any desired proportion of cells. For example, it may be desirable to provide only engineered CD8+ cells to a patient, only engineered CD4+ cells to a patient, or a desired ratio of CD4+ to CD8+ cells, such as equal numbers of CD4+ and CD8+ cells.

In some embodiments, PBMCs are used directly for genetic modification with the immune cells (such as CARs) using methods as described herein. In some embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8⁺ cells are further sorted into naive, central memory, and effector cells by identifying cell surface antigens that are associated with each of these types of CD8⁺ cells. In some embodiments, the expression of phenotypic markers of central memory T cells includes CD3, CD28, CD44, CD45RO, CD45RA and CD127 and are negative for granzyme B. In some embodiments, central memory T cells are CD3⁺, CD28⁺, CD44^{hi}, CD45RO^{hi}, CD45RA^{low} and CD127^{hi} CD8⁺ T cells. In some embodiments, effector T cells are negative for CD62L, CCR7, CD28, and CD127 and positive for granzyme B and perforin. In some embodiments, CD4⁺ T cells are further sorted into subpopulations. For example, CD4⁺ T helper cells may be sorted into naive, central memory, and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead, tissue culture bag, plate, flask or other surface, in a culture medium with appropriate cytokines, such as IL-2, IL-7 and/or IL-15. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In some embodiments, the T cells are obtained from a donor subject. In some embodiments, the donor subject is human patient afflicted with melanoma. In some embodiments, the T cells are derived from pluripotent stem cells maintained under conditions favorable to the differentiation of the stem cells to T cells.

Other aspects of the present invention are directed to compositions comprising a polynucleotide provided herein, a vector provided herein, a polypeptide provided herein, or an in vitro cell provided herein.

In some embodiments, the composition is a pharmaceutical composition further comprising a pharmaceutically acceptable carrier, diluent, solubilizer, emulsifier, excipient, preservative and/or adjuvant.

In some embodiments, the composition is selected for parenteral delivery. The preparation of such pharmaceutically acceptable compositions is within the ability of one skilled in the art. In some embodiments, buffers are used to maintain the composition at physiological pH or at a slightly lower pH, typically within a pH range of from about 5 to about 8. In some embodiments, when parenteral administration is contemplated, the composition is in the form of a pyrogen-free, parenterally acceptable aqueous solution comprising a desired CAR comprising an antigen binding domain that specifically binds to MART-1, with or without additional therapeutic agents, in a pharmaceutically acceptable vehicle.

In some embodiments the vehicle for parenteral injection is sterile distilled water in which a CAR, with at least one additional therapeutic agent, is formulated as a sterile, isotonic solution, properly preserved.

In some embodiments, the preparation involves the formulation of the desired CAR with polymeric compounds (such as polylactic acid or polyglycolic acid), beads or liposomes, that provide for the controlled or sustained release of the product, which are then be delivered via a depot injection. In some embodiments, implantable drug delivery devices are used to introduce the desired molecule.

In some embodiments, the composition includes more than one CAR, e.g., CARs directed to different antigens and CARs directed to the same antigen (e.g., MART-1) but to a different region of the antigen. An example of the latter includes CARS derived from different antibodies.

### VI. Methods for Manufacturing CAR-Expressing Cells

Another aspect of the present invention is directed to a method of making (manufacturing) a cell expressing a CAR. The method comprises transducing a cell with a polynucleotide of the present invention under suitable conditions. In some embodiments, the method comprises transducing a cell with a polynucleotide encoding a CAR, wherein the CAR comprises an antigen binding domain that specifically binds to MART-1, e.g., an extracellular epitope of MART-1. In some embodiments, the method comprises transducing a cell with a vector comprising a polynucleotide encoding a CAR, wherein the CAR comprises an antigen binding domain that specifically binds to MART-1, e.g., an extracellular epitope of MART-1. In some embodiments, the method further comprises isolating the cell.

The manufacturing methods do not require selecting and/or separating transduced T cells for expression of CD4 or CD8. Instead, flow characteristics may be used to detect a composition's percentage of CD4+ cells and CD8+ cells; however, no selection is needed. Accordingly, rather than taking about twenty-one days to manufacture a composition of transduced T cells, the present invention only requires about six days. Thus, in about a week, a patient may be transfused with his/her T cells that have been engineered to express an anti-MART-1 CAR rather than after about three weeks. Examples of CAR T cell manufacturing methods are described in U.S. Patent Publication No. 2015/0344844, which is hereby incorporated by reference in its entirety.

### VII. Cancer Treatments

The methods of the present invention may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In some embodiments, the methods induce a complete response. In other embodiments, the methods induce a partial response.

In some embodiments, the method comprises administering to a subject an effective amount of a cell comprising a polynucleotide encoding a CAR, wherein the CAR comprises an antigen binding domain that specifically binds to MART-1, e.g., an extracellular epitope of MART-1. In some embodiments, the method comprises administering to a subject an effective amount of a cell comprising a vector comprising a polynucleotide encoding a CAR, wherein the CAR comprises an antigen binding domain that specifically binds to MART-1, e.g., an extracellular epitope of MART-1. In some embodiments, the method comprises administering to a subject an effective amount of a cell comprising a CAR encoded by a polynucleotide of the present invention, wherein the CAR comprises an antigen binding domain that specifically binds to MART-1, e.g., an extracellular epitope of MART-1.

Some embodiments relate to a method of inducing an immune response in a subject comprising administering an effective amount of the engineered immune cells of the present application. In some embodiments, the immune response is a T cell-mediated immune response. In some embodiments, the T cell-mediated immune response is directed against one or more target cells. In some embodiments, the engineered immune cell comprises a CAR, such as those provided herein. In some embodiments, the target cell is a tumor cell, e.g., a melanoma cell.

Some embodiments relate to a method for treating or preventing melanoma, said method comprising administering to a subject in need thereof an effective amount of one engineered cell type, e.g., a T cell, or a composition comprising a plurality of said cells, wherein the engineered cells comprise at least one CAR comprising antigen binding domain that specifically binds to MART-1, e.g., an extracellular epitope of MART-1.

In some embodiments, the methods of treating a cancer in a subject in need thereof comprise a T cell therapy. In one embodiment, the T cell therapy of the present invention is Engineered Autologous Cell Therapy (eACT™). According to this embodiment, the method may include collecting blood cells from the patient. The isolated blood cells (e.g., T cells) may then be engineered to express an anti-MART-1 CAR of the present invention (“anti-MART-1 CAR T cells”). In some embodiments, the anti-MART-1 CAR T cells are administered to the patient. In some embodiments, the anti-MART-1 CAR T cells treat a tumor or a cancer, e.g., a melanoma, in the patient. In one embodiment the anti-MART-1 CAR T cells reduce the size of a tumor or a cancer, e.g. melanoma.

In some embodiments, the donor T cells for use in the T cell therapy are obtained from the patient (e.g., for an autologous T cell therapy). In other embodiments, the donor T cells for use in the T cell therapy are obtained from a subject that is not the patient (e.g., allogeneic T cell therapy).

The T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10⁹ cells, at least about 10¹⁰ cells, or at least about 10¹¹ cells. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the anti-MART-1 CAR T cells is about 1×10⁵ cells/kg, 2×10⁵ cells/kg, 3×10⁵ cells/kg, 4×10⁵ cells/kg, 5×10⁵ cells/kg, 1×10⁶ cells/kg, 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, the methods further comprise administering (separately or together with cells or compositions of the present invention) a chemotherapeutic. In some embodiments, the chemotherapeutic selected from Dacarbazine (also called DTIC), Temozolomide, Nab-paclitaxel, Paclitaxel, Cisplatin, Carboplatin, and Vinblastine. In some embodiments, the chemotherapeutic agent is administered at the same time or within one week after the administration of the engineered cell or composition. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or composition. In some embodiments, the chemotherapeutic agent is administered at least 1 month before administering the cell or nucleic acid. In some embodiments, the methods further comprise administering two or more chemotherapeutic agents, such as a checkpoint inhibitor.

Examples of treatment methods are disclosed in U.S. Pat. No. 9,855,298 and WO2016019755, each of which are hereby incorporated by reference in their entirety.

Although methods and materials similar or equivalent to those described herein may be used in the practice or testing of the present invention, suitable methods and materials are described below. All publications, patent applications, patents, and other references mentioned herein are incorporated by reference in their entirety. The references cited herein are not admitted to be prior art to the claimed invention. In addition, the materials, methods, and examples are illustrative only and are not intended to be limiting.

1. A polynucleotide encoding a chimeric antigen receptor (CAR), wherein the CAR comprises at least an antigen binding domain, an activation domain, and a co-stimulatory domain, wherein the antigen binding domain is specific to MART-1.

2. (canceled)

3. The polynucleotide of claim 1, wherein the antigen binding domain comprises an antibody or an antigen binding fragment thereof selected from the group consisting of an IgG, an Fab, an Fab′, an F(ab′)₂, an Fv, an scFv, and a single-domain antibody (dAB).

4-9. (canceled)

10. The polynucleotide of claim 39, comprising a VL complementarity determining region (CDR) 1 (VL CDR1), a VL CDR2, and a VL CDR3, wherein the VL CDR1 is at least 90% identical to SEQ ID NO: 1, the VL CDR2 is at least 90% identical to SEQ ID NO: 2, and the VL CDR3 is at least 90% identical to SEQ ID NO: 3.

11. The polynucleotide of claim 3, comprising a VH complementarity determining region (CDR) 1 (VH CDR1), a VH CDR2, and a VH CDR3, wherein the VH CDR1 is at least 90% identical to SEQ ID NO: 7 or 10, the VH CDR2 is at least 90% identical to SEQ ID NO: 8 or 11, and the VH CDR3 is at least 90% identical to SEQ ID NO: 9.

12-13. (canceled)

14. The polynucleotide of claim 1, wherein the antigen binding domain is at least 80% identical to SEQ ID NO: 20.

15. The polynucleotide of claim 1, wherein the antigen binding domain is at least 80% identical to SEQ ID NO: 21.

16-17. (canceled)

18. The polynucleotide of claim 1, wherein the antigen binding domain is encoded by a polynucleotide that is at least 80% identical to SEQ ID NO: 28.

19. The polynucleotide of claim 1, wherein the antigen binding domain is encoded by a polynucleotide that is at least 80% identical to SEQ ID NO: 29.

20. The polynucleotide of claim 3 comprising a VL complementarity determining region (CDR) 1 (VL CDR1), a VL CDR2, and a VL CDR3, wherein the VL CDR1 is at least 90% identical to SEQ ID NO: 4, the VL CDR2 is at least 90% identical to SEQ ID NO: 5, and the VL CDR3 is at least 90% identical to SEQ ID NO: 6.

21. The polynucleotide of claim 3 comprising a VH complementarity determining region (CDR) 1 (VH CDR1), a VH CDR2, and a VH CDR3, wherein the VH CDR1 is at least 90% identical to SEQ ID NO: 12, 15, or 17, the VH CDR2 is at least 90% identical to SEQ ID NO: 13 or 16, and the VH CDR3 is at least 90% identical to SEQ ID NO: 14.

22-23. (canceled)

24. The polynucleotide of claim 1, wherein the antigen binding domain is at least 80% identical to SEQ ID NO: 24.

25. The polynucleotide of claim 1, wherein the antigen binding domain is at least 80% identical to SEQ ID NO: 25.

26-27. (canceled)

28. The polynucleotide of claim 1, wherein the antigen binding domain is encoded by a polynucleotide that is at least 80% identical to SEQ ID NO: 32.

29. The polynucleotide of claim 1, wherein the antigen binding domain is encoded by a polynucleotide that is at least 80% identical to SEQ ID NO: 33.

30-31. (canceled)

32. A vector comprising the polynucleotide of claim 1.

33-35. (canceled)

36. A chimeric antigen receptor (CAR) encoded by the polynucleotide of claim 1.

37. A cell comprising the polynucleotide of claim 1.

38-50. (canceled)

51. A method for manufacturing a cell expressing a chimeric antigen receptor (CAR), comprising a step of transducing a cell with the polynucleotide of claim 1.

52-57. (canceled)

58. A method for treating melanoma comprising administering to a subject in need thereof the cell of claim 37.

59. A method for treating melanoma comprising administering to a subject in need thereof a cell expressing a chimeric antigen receptor (CAR) that specifically targets MART-1.

60-75. (canceled)

76. The polynucleotide of claim 1, wherein the costimulatory comprises CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

77. The polynucleotide of claim 76, wherein the costimulatory domain comprises CD28, CD134 (OX40), CD137 (4-1BB) and fragments or combinations thereof.

78. The polynucleotide of claim 1, wherein the activation domain comprises CD3z and fragments thereof.


SEQUENCE LISTING (94 sequences)

SEQ ID NO: 1 - protein, 11 aa, Artificial Sequence
  Description of Artificial Sequence Synthetic peptide
SASQGIHNYLN

SEQ ID NO: 2 - protein, 7 aa, Artificial Sequence
  Description of Artificial Sequence Synthetic peptide
YTSSLHS

SEQ ID NO: 3 - protein, 9 aa, Artificial Sequence
  Description of Artificial Sequence Synthetic peptide
QQYSKLPRT

SEQ ID NO: 4 - protein, 13 aa, Artificial Sequence
  Description of Artificial Sequence Synthetic peptide
QASQSVYKNNRLS

SEQ ID NO: 5 - protein, 7 aa, Artificial Sequence
  Description of Artificial Sequence Synthetic peptide
GASTLAS

SEQ ID NO: 6 - protein, 10 aa, Artificial Sequence
  Description of Artificial Sequence Synthetic peptide
AGEYNNMLYP

SEQ ID NO: 7 - protein, 12 aa, Artificial Sequence
  Description of Artificial Sequence Synthetic peptide
GFSLNTSGMNVG

SEQ ID NO: 8 - protein, 16 aa, Artificial Sequence
  Description of Artificial Sequence Synthetic peptide
HIWWNDDKYYNPALKS

SEQ ID NO: 9 - protein, 12 aa, Artificial Sequence
  Description of Artificial Sequence Synthetic peptide
SYFGDYVWYFDV

SEQ ID NO: 10 - protein, 9 aa, Artificial Sequence
  Description of Artificial Sequence Synthetic peptide
GFSLNTSGM

SEQ ID NO: 11 - protein, 5 aa, Artificial Sequence
  Description of Artificial Sequence Synthetic peptide
WWNDD

SEQ ID NO: 12 - protein, 5 aa, Artificial Sequence
  Description of Artificial Sequence Synthetic peptide
SPVMI

SEQ ID NO: 13 - protein, 14 aa, Artificial Sequence
  Description of Artificial Sequence Synthetic peptide
IISISGNTGYASWA

SEQ ID NO: 14 - protein, 13 aa, Artificial Sequence
  Description of Artificial Sequence Synthetic peptide
MGYDSSSGYAWNL

SEQ ID NO: 15 - protein, 7 aa, Artificial Sequence
  Description of Artificial Sequence Synthetic peptide
GFSISSP

SEQ ID NO: 16 - protein, 3 aa, Artificial Sequence
  Description of Artificial Sequence Synthetic peptide
SIS

SEQ ID NO: 17 - protein, 10 aa, Artificial Sequence
  Description of Artificial Sequence Synthetic peptide
GFSISSPVMI

SEQ ID NO: 18 - protein, 108 aa, Artificial Sequence
  Description of Artificial Sequence Synthetic polypeptide
DIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKR

SEQ ID NO: 19 - protein, 122 aa, Artificial Sequence
  Description of Artificial Sequence Synthetic polypeptide
QVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSS

SEQ ID NO: 20 - protein, 482 aa, Artificial Sequence
  Description of Artificial Sequence Synthetic polypeptide
MALPVTALLLPLALLLHAARPQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSGSTSGSGKPGSGEGSTKGDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 482 aa, Artificial Sequence
  Description of Artificial Sequence Synthetic polypeptide
MALPVTALLLPLALLLHAARPDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRGSTSGSGKPGSGEGSTKGQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 111 aa, Artificial Sequence
  Description of Artificial Sequence Synthetic polypeptide
QIVMTQTPASVSAAVGGTVTINCQASQSVYKNNRLSWFQQKPGQPPKLLIYGASTLASGVPSRFKGSGSGTQFTLTISDVQCDDAATYYCAGEYNNMLYPFGGGTVVVVKG

SEQ ID NO: 23 - protein, 118 aa, Artificial Sequence
  Description of Artificial Sequence Synthetic polypeptide
QSVEEPGGRLVTPGTPLTLTCTVSGFSISSPVMIWVRQAPEKGLEYIGIISISGNTGYASWAKGRFTISKTTTTVDLKITSPTTEDTATYFCARMGYDSSSGYAWNLWGPGTLVTVSS

SEQ ID NO: 24 - protein, 481 aa, Artificial Sequence
  Description of Artificial Sequence Synthetic polypeptide
MALPVTALLLPLALLLHAARPQSVEEPGGRLVTPGTPLTLTCTVSGFSISSPVMIWVRQAPEKGLEYIGIISISGNTGYASWAKGRFTISKTTTTVDLKITSPTTEDTATYFCARMGYDSSSGYAWNLWGPGTLVTVSSGSTSGSGKPGSGEGSTKGQIVMTQTPASVSAAVGGTVTINCQASQSVYKNNRLSWFQQKPGQPPKLLIYGASTLASGVPSRFKGSGSGTQFTLTISDVQCDDAATYYCAGEYNNMLYPFGGGTVVVVKGAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 481 aa, Artificial Sequence
  Description of Artificial Sequence Synthetic polypeptide
MALPVTALLLPLALLLHAARPQIVMTQTPASVSAAVGGTVTINCQASQSVYKNNRLSWFQQKPGQPPKLLIYGASTLASGVPSRFKGSGSGTQFTLTISDVQCDDAATYYCAGEYNNMLYPFGGGTVVVVKGGSTSGSGKPGSGEGSTKGQSVEEPGGRLVTPGTPLTLTCTVSGFSISSPVMIWVRQAPEKGLEYIGIISISGNTGYASWAKGRFTISKTTTTVDLKITSPTTEDTATYFCARMGYDSSSGYAWNLWGPGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 324 bp, Artificial Sequence
  Description of Artificial Sequence Synthetic polynucleotide
gacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcgg

SEQ ID NO: 27 - DNA, 366 bp, Artificial Sequence
  Description of Artificial Sequence Synthetic polynucleotide
caggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagc

SEQ ID NO: 28 - DNA, 1446 bp, Artificial Sequence
  Description of Artificial Sequence Synthetic polynucleotide
atggcactccccgtaactgctctgctgctgccgttggcattgctcctgcacgccgcacgcccgcaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcgggagtacgtccggctcaggtaaacctggaagtggggaaggatcaacgaaaggcgacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcgggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 29 - DNA, 1446 bp, Artificial Sequence
  Description of Artificial Sequence Synthetic polynucleotide
atggcactccccgtaactgctctgctgctgccgttggcattgctcctgcacgccgcacgcccggacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcgggggagtacgtccggctcaggtaaacctggaagtggggaaggatcaacgaaaggccaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcgccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 30 - DNA, 333 bp, Artificial Sequence
  Description of Artificial Sequence Synthetic polynucleotide
cagattgtgatgactcaaacacccgcctctgtttccgccgccgttggcggcaccgtcaccattaactgccaggcaagtcaatccgtttataaaaacaacagactgagttggtttcagcagaagccaggacagccacctaaacttctgatttacggcgcttcaactctggcatccggggtccccagcagattcaagggctctggctccgggacgcagttcactctgactatatctgatgtccagtgcgatgacgccgctacatactactgtgccggcgaatacaataatatgctctatcctttcggcggcgggacagtggtcgtggtcaaaggc

SEQ ID NO: 31 - DNA, 354 bp, Artificial Sequence
  Description of Artificial Sequence Synthetic polynucleotide
cagagtgtcgaagaacctggtgggaggctggtgacccctggaactccactgacactgacgtgtacagtgagcggttttagcatttcttcccctgtcatgatttgggttagacaggcgcccgaaaagggactggaatacatcggtataatcagtatctccggaaataccggttacgcctcatgggcgaagggtcgatttaccattagcaaaacaactaccaccgtagatcttaagatcacaagccccactacagaggatacagccacttacttttgcgcacgaatgggctatgattccagctcaggctatgcatggaacctctggggtccggggacgctggtcaccgtgtcctca

SEQ ID NO: 32 - DNA, 1443 bp, Artificial Sequence
  Description of Artificial Sequence Synthetic polynucleotide
atggcactccccgtaactgctctgctgctgccgttggcattgctcctgcacgccgcacgcccgcagagtgtcgaagaacctggtgggaggctggtgacccctggaactccactgacactgacgtgtacagtgagcggttttagcatttcttcccctgtcatgatttgggttagacaggcgcccgaaaagggactggaatacatcggtataatcagtatctccggaaataccggttacgcctcatgggcgaagggtcgatttaccattagcaaaacaactaccaccgtagatcttaagatcacaagccccactacagaggatacagccacttacttttgcgcacgaatgggctatgattccagctcaggctatgcatggaacctctggggtccggggacgctggtcaccgtgtcctcaggttccactagtggatctggtaaacctggatcaggtgaaggctcaaccaagggtcagattgtgatgactcaaacacccgcctctgtttccgccgccgttggcggcaccgtcaccattaactgccaggcaagtcaatccgtttataaaaacaacagactgagttggtttcagcagaagccaggacagccacctaaacttctgatttacggcgcttcaactctggcatccggggtccccagcagattcaagggctctggctccgggacgcagttcactctgactatatctgatgtccagtgcgatgacgccgctacatactactgtgccggcgaatacaataatatgctctatcctttcggcggcgggacagtggtcgtggtcaaaggcgccgctgctcttgacaacgagaaatctaacgggaccattatccatgtgaaaggaaagcacctttgtccgtcaccgttgttccccgggcctagcaagccattttgggtgctcgtcgtggtgggaggcgtgctggcttgctactcattgttggttaccgttgcgtttatcatcttctgggtcagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgc

SEQ ID NO: 33 - DNA, 1443 bp, Artificial Sequence
  Description of Artificial Sequence Synthetic polynucleotide
atggcactccccgtaactgctctgctgctgccgttggcattgctcctgcacgccgcacgcccgcagatcgtgatgacacagacccccgcatccgtaagcgctgctgttggtggcacagtgactattaactgccaggcgtctcaatctgtttataaaaacaaccgccttagttggtttcagcagaagcctgggcagccacctaaactgctgatttacggggccagcacgttggcaagcggggtaccatctcggtttaaaggctccggttcagggactcaattcaccttgacaatctccgatgtgcagtgcgacgatgcagcaacatactattgcgcaggggagtataataatatgctgtacccatttggaggcgggactgtggtggttgttaaaggcggctctacctccgggtccggaaagcctggatcaggtgaggggagcacaaaaggccaatctgtcgaggagcccggtggccgcctggtgactcccgggactcctctcaccctgacttgtaccgtcagcggcttcagcattagctccccggtgatgatttgggtgcggcaggcacccgaaaagggcctggaatacatcgggataatcagcatttctggcaatacgggctacgccagttgggccaaaggcagatttactatctctaaaaccacaaccacagttgatttgaagatcaccagtcctacaaccgaggatacagccacgtatttttgcgcacgcatgggctacgactctagctctggttatgcctggaacctgtggggacctggtacccttgttacagtctctagtgctgcagcgctcgataatgagaagtccaatggtacaatcattcacgtgaagggtaaacatctttgtccttcacccctcttcccgggacctagcaagccgttctgggttctcgtcgtggtgggcggcgttctggcctgctatagcctgctcgttacggtagcgttcattatcttttgggttagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgc

SEQ ID NO: 34 - DNA, 63 bp, Artificial Sequence
  Description of Artificial Sequence Synthetic oligonucleotide
atggcactccccgtaactgctctgctgctgccgttggcattgctcctgcacgccgcacgcccg

SEQ ID NO: 35 - protein, 21 aa, Artificial Sequence
  Description of Artificial Sequence Synthetic peptide
MALPVTALLLPLALLLHAARP

SEQ ID NO: 36 - DNA, 54 bp, Artificial Sequence
  Description of Artificial Sequence Synthetic oligonucleotide
gggagtacgtccggctcaggtaaacctggaagtggggaaggatcaacgaaaggc

SEQ ID NO: 37 - protein, 18 aa, Artificial Sequence
  Description of Artificial Sequence Synthetic peptide
GSTSGSGKPGSGEGSTKG

SEQ ID NO: 38 - DNA, 294 bp, Artificial Sequence
  Description of Artificial Sequence Synthetic polynucleotide
cttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagc

SEQ ID NO: 39 - protein, 98 aa, Artificial Sequence
  Description of Artificial Sequence Synthetic polypeptide
LDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 40 - DNA, 90 bp, Artificial Sequence
  Description of Artificial Sequence Synthetic oligonucleotide
cttgataatgaaaagtcaaacggaacaatcattcacgtgaagggcaagcacctctgtccgtcacccttgttccctggtccatccaagcca

SEQ ID NO: 41 - protein, 30 aa, Artificial Sequence
  Description of Artificial Sequence Synthetic polypeptide
LDNEKSNGTIIHVKGKHLCPSPLFPGPSKP

SEQ ID NO: 42 - DNA, 81 bp, Artificial Sequence
  Description of Artificial Sequence Synthetic oligonucleotide
ttctgggtgttggtcgtagtgggtggagtcctcgcttgttactctctgctcgtcaccgtggcttttataatcttctgggtt

SEQ ID NO: 43 - protein, 27 aa, Artificial Sequence
  Description of Artificial Sequence Synthetic peptide
FWVLVVVGGVLACYSLLVTVAFIIFWV

SEQ ID NO: 44 - DNA, 123 bp, Artificial Sequence
  Description of Artificial Sequence Synthetic polynucleotide
agatccaaaagaagccgcctgctccatagcgattacatgaatatgactccacgccgccctggccccacaaggaaacactaccagccttacgcaccacctagagatttcgctgcctatcggagc

SEQ ID NO: 45 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 46 - DNA, 336 bp, Artificial Sequence
  Description of Artificial Sequence Synthetic polynucleotide
agggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 47 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 336 bp, Artificial Sequence
  Description of Artificial Sequence Synthetic polynucleotide
agggtgaagttttccagatctgcagatgcaccagcgtataagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 49 - protein, 112 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - DNA, 117 bp, Artificial Sequence
  Description of Artificial Sequence Synthetic polynucleotide
attgaggtgatgtatccaccgccttacctggataacgaaaagagtaacggtaccatcattcacgtgaaaggtaaacacctgtgtccttctcccctcttccccgggccatcaaagccc

SEQ ID NO: 51 - protein, 39 aa, Artificial Sequence
  Description of Artificial Sequence Synthetic polypeptide
IEVMYPPPYLDNEKSNGTIIHVKGKHLCPSPLFPGPSKP

SEQ ID NO: 52 - DNA, 288 bp, Artificial Sequence
  Description of Artificial Sequence Synthetic polynucleotide
gctgcagcattgagcaactcaataatgtattttagtcactttgtaccagtgttcttgccggctaagcctactaccacacccgctccacggccacctaccccagctcctaccatcgcttcacagcctctgtccctgcgcccagaggcttgccgaccggccgcagggggcgctgttcataccagaggactggatttcgcctgcgatatctatatctgggcacccctggccggaacctgcggcgtactcctgctgtccctggtcatcacgctctattgtaatcacaggaac

SEQ ID NO: 53 - protein, 96 aa, Artificial Sequence
  Description of Artificial Sequence Synthetic polypeptide
AAALSNSIMYFSHFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 54 - DNA, 1452 bp, Artificial Sequence
  Description of Artificial Sequence Synthetic polynucleotide
atggcactccccgtaactgctctgctgctgccgttggcattgctcctgcacgccgcacgcccgcagatcgtgatgacacagacccccgcatccgtaagcgctgctgttggtggcacagtgactattaactgccaggcgtctcaatctgtttataaaaacaaccgccttagttggtttcagcagaagcctgggcagccacctaaactgctgatttacggggccagcacgttggcaagcggggtaccatctcggtttaaaggctccggttcagggactcaattcaccttgacaatctccgatgtgcagtgcgacgatgcagcaacatactattgcgcaggggagtataataatatgctgtacccatttggaggcgggactgtggtggttgttaaaggcggctctacctccgggtccggaaagcctggatcaggtgaggggagcacaaaaggccaatctgtcgaggagcccggtggccgcctggtgactcccgggactcctctcaccctgacttgtaccgtcagcggcttcagcattagctccccggtgatgatttgggtgcggcaggcacccgaaaagggcctggaatacatcgggataatcagcatttctggcaatacgggctacgccagttgggccaaaggcagatttactatctctaaaaccacaaccacagttgatttgaagatcaccagtcctacaaccgaggatacagccacgtatttttgcgcacgcatgggctacgactctagctctggttatgcctggaacctgtggggacctggtacccttgttacagtctctagtggagggggtggaagtctcgataatgagaagtccaatggtacaatcattcacgtgaagggtaaacatctttgtccttcacccctcttcccgggacctagcaagccgttctgggttctcgtcgtggtgggcggcgttctggcctgctatagcctgctcgttacggtagcgttcattatcttttgggttagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 55 - protein, 483 aa, Artificial Sequence
  Description of Artificial Sequence Synthetic polypeptide
MALPVTALLLPLALLLHAARPQIVMTQTPASVSAAVGGTVTINCQASQSVYKNNRLSWFQQKPGQPPKLLIYGASTLASGVPSRFKGSGSGTQFTLTISDVQCDDAATYYCAGEYNNMLYPFGGGTVVVVKGGSTSGSGKPGSGEGSTKGQSVEEPGGRLVTPGTPLTLTCTVSGFSISSPVMIWVRQAPEKGLEYIGIISISGNTGYASWAKGRFTISKTTTTVDLKITSPTTEDTATYFCARMGYDSSSGYAWNLWGPGTLVTVSSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 1467 bp, Artificial Sequence
  Description of Artificial Sequence Synthetic polynucleotide
atggcactccccgtaactgctctgctgctgccgttggcattgctcctgcacgccgcacgcccgcagatcgtgatgacacagacccccgcatccgtaagcgctgctgttggtggcacagtgactattaactgccaggcgtctcaatctgtttataaaaacaaccgccttagttggtttcagcagaagcctgggcagccacctaaactgctgatttacggggccagcacgttggcaagcggggtaccatctcggtttaaaggctccggttcagggactcaattcaccttgacaatctccgatgtgcagtgcgacgatgcagcaacatactattgcgcaggggagtataataatatgctgtacccatttggaggcgggactgtggtggttgttaaaggcggctctacctccgggtccggaaagcctggatcaggtgaggggagcacaaaaggccaatctgtcgaggagcccggtggccgcctggtgactcccgggactcctctcaccctgacttgtaccgtcagcggcttcagcattagctccccggtgatgatttgggtgcggcaggcacccgaaaagggcctggaatacatcgggataatcagcatttctggcaatacgggctacgccagttgggccaaaggcagatttactatctctaaaaccacaaccacagttgatttgaagatcaccagtcctacaaccgaggatacagccacgtatttttgcgcacgcatgggctacgactctagctctggttatgcctggaacctgtggggacctggtacccttgttacagtctctagtggagggggtggaagtgggggcggtggcagcctcgataatgagaagtccaatggtacaatcattcacgtgaagggtaaacatctttgtccttcacccctcttcccgggacctagcaagccgttctgggttctcgtcgtggtgggcggcgttctggcctgctatagcctgctcgttacggtagcgttcattatcttttgggttagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 57 - protein, 488 aa, Artificial Sequence
  Description of Artificial Sequence Synthetic polypeptide
MALPVTALLLPLALLLHAARPQIVMTQTPASVSAAVGGTVTINCQASQSVYKNNRLSWFQQKPGQPPKLLIYGASTLASGVPSRFKGSGSGTQFTLTISDVQCDDAATYYCAGEYNNMLYPFGGGTVVVVKGGSTSGSGKPGSGEGSTKGQSVEEPGGRLVTPGTPLTLTCTVSGFSISSPVMIWVRQAPEKGLEYIGIISISGNTGYASWAKGRFTISKTTTTVDLKITSPTTEDTATYFCARMGYDSSSGYAWNLWGPGTLVTVSSGGGGSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1482 bp, Artificial Sequence
  Description of Artificial Sequence Synthetic polynucleotide
atggcactccccgtaactgctctgctgctgccgttggcattgctcctgcacgccgcacgcccgcagatcgtgatgacacagacccccgcatccgtaagcgctgctgttggtggcacagtgactattaactgccaggcgtctcaatctgtttataaaaacaaccgccttagttggtttcagcagaagcctgggcagccacctaaactgctgatttacggggccagcacgttggcaagcggggtaccatctcggtttaaaggctccggttcagggactcaattcaccttgacaatctccgatgtgcagtgcgacgatgcagcaacatactattgcgcaggggagtataataatatgctgtacccatttggaggcgggactgtggtggttgttaaaggcggctctacctccgggtccggaaagcctggatcaggtgaggggagcacaaaaggccaatctgtcgaggagcccggtggccgcctggtgactcccgggactcctctcaccctgacttgtaccgtcagcggcttcagcattagctccccggtgatgatttgggtgcggcaggcacccgaaaagggcctggaatacatcgggataatcagcatttctggcaatacgggctacgccagttgggccaaaggcagatttactatctctaaaaccacaaccacagttgatttgaagatcaccagtcctacaaccgaggatacagccacgtatttttgcgcacgcatgggctacgactctagctctggttatgcctggaacctgtggggacctggtacccttgttacagtctctagtggagggggtggaagtgggggcggtggcagcggcggtggcggcagtctcgataatgagaagtccaatggtacaatcattcacgtgaagggtaaacatctttgtccttcacccctcttcccgggacctagcaagccgttctgggttctcgtcgtggtgggcggcgttctggcctgctatagcctgctcgttacggtagcgttcattatcttttgggttagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 59 - protein, 493 aa, Artificial Sequence
  Description of Artificial Sequence Synthetic polypeptide
MALPVTALLLPLALLLHAARPQIVMTQTPASVSAAVGGTVTINCQASQSVYKNNRLSWFQQKPGQPPKLLIYGASTLASGVPSRFKGSGSGTQFTLTISDVQCDDAATYYCAGEYNNMLYPFGGGTVVVVKGGSTSGSGKPGSGEGSTKGQSVEEPGGRLVTPGTPLTLTCTVSGFSISSPVMIWVRQAPEKGLEYIGIISISGNTGYASWAKGRFTISKTTTTVDLKITSPTTEDTATYFCARMGYDSSSGYAWNLWGPGTLVTVSSGGGGSGGGGSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 1455 bp, Artificial Sequence
  Description of Artificial Sequence Synthetic polynucleotide
atggcactccccgtaactgctctgctgctgccgttggcattgctcctgcacgccgcacgcccgcaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcgggagtacgtccggctcaggtaaacctggaagtggggaaggatcaacgaaaggcgacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcggggagggggtggaagtcttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 61 - protein, 484 aa, Artificial Sequence
  Description of Artificial Sequence Synthetic polypeptide
MALPVTALLLPLALLLHAARPQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSGSTSGSGKPGSGEGSTKGDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1470 bp, Artificial Sequence
  Description of Artificial Sequence Synthetic polynucleotide
atggcactccccgtaactgctctgctgctgccgttggcattgctcctgcacgccgcacgcccgcaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcgggagtacgtccggctcaggtaaacctggaagtggggaaggatcaacgaaaggcgacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcggggagggggtggaagtgggggcggtggcagccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 63 - protein, 489 aa, Artificial Sequence
  Description of Artificial Sequence Synthetic polypeptide
MALPVTALLLPLALLLHAARPQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSGSTSGSGKPGSGEGSTKGDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRGGGGSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 1485 bp, Artificial Sequence
  Description of Artificial Sequence Synthetic polynucleotide
atggcactccccgtaactgctctgctgctgccgttggcattgctcctgcacgccgcacgcccgcaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcgggagtacgtccggctcaggtaaacctggaagtggggaaggatcaacgaaaggcgacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcggggagggggtggaagtgggggcggtggcagcggcggtggcggcagtcttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 65 - protein, 494 aa, Artificial Sequence
  Description of Artificial Sequence Synthetic polypeptide
MALPVTALLLPLALLLHAARPQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSGSTSGSGKPGSGEGSTKGDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRGGGGSGGGGSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 1455 bp, Artificial Sequence
  Description of Artificial Sequence Synthetic polynucleotide
atggcactccccgtaactgctctgctgctgccgttggcattgctcctgcacgccgcacgcccggacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcgggggagtacgtccggctcaggtaaacctggaagtggggaaggatcaacgaaaggccaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcggagggggtggaagtcttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 67 - protein, 484 aa, Artificial Sequence
  Description of Artificial Sequence Synthetic polypeptide
MALPVTALLLPLALLLHAARPDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRGSTSGSGKPGSGEGSTKGQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1470 bp, Artificial Sequence
  Description of Artificial Sequence Synthetic polynucleotide
atggcactccccgtaactgctctgctgctgccgttggcattgctcctgcacgccgcacgcccggacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcgggggagtacgtccggctcaggtaaacctggaagtggggaaggatcaacgaaaggccaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcggagggggtggaagtgggggcggtggcagccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 69 - protein, 489 aa, Artificial Sequence
  Description of Artificial Sequence Synthetic polypeptide
MALPVTALLLPLALLLHAARPDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRGSTSGSGKPGSGEGSTKGQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSGGGGSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - DNA, 1485 bp, Artificial Sequence
  Description of Artificial Sequence Synthetic polynucleotide
atggcactccccgtaactgctctgctgctgccgttggcattgctcctgcacgccgcacgcccggacatacaaatgacccagacaacgtcaagtctgtctgcgtccttgggggacagggtcactatttcttgctccgcgagtcaagggatacacaattatcttaattggtaccaacagaagccggacggcactgtcaaattgttgatatactacaccagcagccttcactcaggagttccctcccgctttagcggatccggatctggcacggattacagccttacaatctctaatctggagcctgaggacattgcaacatatttttgccagcaatatagtaagctccctcgcacgttcggcggaggtacaaaattggagataaagcgggggagtacgtccggctcaggtaaacctggaagtggggaaggatcaacgaaaggccaggttaccttgaaggaaagcggtcctggtatccttcagccatcccagactctcagcttgacgtgctctttttccggattctccttgaacacgagcggtatgaatgttggatggattagacagccttccggtaaagggctggactggttggcgcacatatggtggaatgacgataagtattacaatcctgcgctgaaaagtaggttgactatatctaaggacacatctaataaccaggtattcctgaaaatagcatcagtcgtaacggccgatactgcgacttattactgtgtccgatcttattttggggattatgtctggtattttgatgtttggggagctgggaccacggtcacagtgtcaagcggagggggtggaagtgggggcggtggcagcggcggtggcggcagtcttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 71 - protein, 494 aa, Artificial Sequence
  Description of Artificial Sequence Synthetic polypeptide
MALPVTALLLPLALLLHAARPDIQMTQTTSSLSASLGDRVTISCSASQGIHNYLNWYQQKPDGTVKLLIYYTSSLHSGVPSRFSGSGSGTDYSLTISNLEPEDIATYFCQQYSKLPRTFGGGTKLEIKRGSTSGSGKPGSGEGSTKGQVTLKESGPGILQPSQTLSLTCSFSGFSLNTSGMNVGWIRQPSGKGLDWLAHIWWNDDKYYNPALKSRLTISKDTSNNQVFLKIASVVTADTATYYCVRSYFGDYVWYFDVWGAGTTVTVSSGGGGSGGGGSGGGGSLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 5 aa, Artificial Sequence
  Description of Artificial Sequence Synthetic peptide
GGGGS

SEQ ID NO: 73 - protein, 18 aa, Artificial Sequence
  Description of Artificial Sequence Synthetic peptide
GSTSGSGKPGSGEGSTKG

SEQ ID NO: 74 - protein, 15 aa, Artificial Sequence
  Description of Artificial Sequence Synthetic peptide
GGGGSGGGGSGGGGS

SEQ ID NO: 75 - protein, 23 aa, Artificial Sequence
  Description of Artificial Sequence Synthetic peptide
  MOD_RES (1)..(3): Any amino acid
  MOD_RES (11)..(13): Any amino acid
  MOD_RES (21)..(23): Any amino acid
XXXGKPGSGEXXXGKPGSGEXXX

SEQ ID NO: 76 - protein, 6 aa, Artificial Sequence
  Description of Artificial Sequence Synthetic peptide
KPGSGE

SEQ ID NO: 77 - protein, 7 aa, Artificial Sequence
  Description of Artificial Sequence Synthetic peptide
GKPGSGE

SEQ ID NO: 78 - protein, 7 aa, Artificial Sequence
  Description of Artificial Sequence Synthetic peptide
GKPGSGG

SEQ ID NO: 79 - protein, 15 aa, Artificial Sequence
  Description of Artificial Sequence Synthetic peptide
GGGSGKPGSGEGGGS

SEQ ID NO: 80 - protein, 16 aa, Artificial Sequence
  Description of Artificial Sequence Synthetic peptide
GGGSGKPGSGEGGGGS

SEQ ID NO: 81 - protein, 15 aa, Artificial Sequence
  Description of Artificial Sequence Synthetic peptide
GGGGSGKPGSGGGGS

SEQ ID NO: 82 - protein, 15 aa, Artificial Sequence
  Description of Artificial Sequence Synthetic peptide
GGGGSGKPGSGEGGS

SEQ ID NO: 83 - protein, 16 aa, Artificial Sequence
  Description of Artificial Sequence Synthetic peptide
GGGGSGKPGSGEGGGS

SEQ ID NO: 84 - protein, 17 aa, Artificial Sequence
  Description of Artificial Sequence Synthetic peptide
GGGGSGKPGSGEGGGGS

SEQ ID NO: 85 - protein, 10 aa, Artificial Sequence
  Description of Artificial Sequence Synthetic peptide
GSGKPGSGEG

SEQ ID NO: 86 - protein, 8 aa, Artificial Sequence
  Description of Artificial Sequence Synthetic peptide
GKPGSGEG

SEQ ID NO: 87 - protein, 8 aa, Artificial Sequence
  Description of Artificial Sequence Synthetic peptide
SGKPGSGE

SEQ ID NO: 88 - protein, 5 aa, Artificial Sequence
  Description of Artificial Sequence Synthetic peptide
KPGSG

SEQ ID NO: 89 - protein, 15 aa, Artificial Sequence
  Description of Artificial Sequence Synthetic peptide
STSGSGKPGSGEGST

SEQ ID NO: 90 - protein, 16 aa, Artificial Sequence
  Description of Artificial Sequence Synthetic peptide
GGGGSGGGGSGGGGSG

SEQ ID NO: 91 - protein, 16 aa, Artificial Sequence
  Description of Artificial Sequence Synthetic peptide
GGGGGSGGGGSGGGGS

SEQ ID NO: 92 - protein, 16 aa, Artificial Sequence
  Description of Artificial Sequence Synthetic peptide
GGGGSGGGGSGGGGGS

SEQ ID NO: 93 - protein, 5 aa, Artificial Sequence
  Description of Artificial Sequence Synthetic peptide
EAAAK

SEQ ID NO: 94 - protein, 7 aa, Artificial Sequence
  Description of Artificial Sequence Synthetic peptide
TSGMNVG